(19) United States
(12) Patent Application Publication
(10) Pub. No.: US 2021/0183486 A1
(43) Pub. Date: Jun. 17, 2021

(54) BIOLOGICAL INFORMATION PROCESSING METHOD, BIOLOGICAL INFORMATION PROCESSING APPARATUS, AND BIOLOGICAL INFORMATION PROCESSING SYSTEM

(71) Applicant: SONY CORPORATION, Tokyo (JP)

(72) Inventors: Kazuhiro SAKURADA, Tokyo (JP); Takayoshi HIRAI, Tokyo (JP); Taro AZUMA, Tokyo (JP); Ryosuke FURUKAWA, Tokyo (JP)

(73) Assignee: SONY CORPORATION, Tokyo (JP)

(21) Appl. No.: 17/251,045

(22) PCT Filed: Jun. 19, 2019
(86) PCT No.: PCT/JP2019/024357

§ 371 Date: Dec. 10, 2020

(30) Foreign Application Priority Data
Jun. 19, 2018 (JP) 2018-116003

(51) Int. Cl. G16H 10/60; G16H 40/20; G16H 50/70; G06F 16/23

(52) U.S. Cl. CPC G16H 10/60; G06F 16/2379; G16H 50/70; G16H 40/20

(57) ABSTRACT

A biological information processing method according to an embodiment includes: acquiring biological information on a subject; based on the biological information, generating condition information representing a biological condition of the subject; and registering the condition information in a P2P database (223) is provided.

### FIELD

The present disclosure relates to a biological information processing method, a biological information processing apparatus, and a biological information processing system.

### BACKGROUND

In recent years, in order to save and share medical data, efforts to record medical data as electronic health records have been made. For example, Patent Literature 1 below discloses recording medical data as an electronic health record and transmitting part of the electronic health record to an EDC (Electronic Data Capture) system.

### CITATION LIST

### Patent Literature

Patent Literature 1: Japanese Unexamined Patent Application Publication No. 2017-208039

Patent Literature 2: Japanese Unexamined Patent Application Publication No. 2012-30038

### SUMMARY

### Technical Problem

Linkage of medical data between each hospital however has not sufficiently progressed. For example, because the electronic health record system differs in each hospital, even when an electronic health record is linked from another hospital, each hospital is not able to appropriately utilize medical data that is recorded in the electronic health record.

The disclosure was thus made in view of the above-described circumstances and the disclosure provides a biological information processing method, a biological information processing apparatus, and a biological information processing system that are new and improved and that enable more appropriate linkage of medical data between each hospital.

### Solution to Problem

For solving the problem described above, a biological information processing method according to one aspect of the present disclosure has acquiring biological information on a subject; based on the biological information, generating condition information representing a biological condition of the subject; and registering the condition information in a P2P database.

For solving the problem described above, a biological information processing apparatus according to one aspect of the present disclosure has a biological information acquisition unit configured to acquire biological information on a subject; a condition information generator configured to generate condition information representing a biological condition of the subject based on the biological information; and a register configure to register the condition information in a P2P database.

For solving the problem described above, a biological information processing system according to one aspect of the present disclosure has a biological information acquisition unit configured to acquire biological information on a subject; a condition information generator configured to generate condition information representing a biological condition of the subject based on the biological information; and a register configure to register the condition information in a P2P database.

For solving the problem described above, a biological information processing method according to one aspect of the present disclosure has acquiring biological information on a subject; generating condition information representing a biological condition of the subject based on the biological information; and registering the condition information as data of a distributed network.

According to the disclosure, it is possible to link condition information that is one type of medical data with each hospital that has a P2P database.

### Advantageous Effects of Invention

As described above, according to the disclosure, it is possible to more appropriately link medical data between each hospital.

Note that the above-described effect is not necessarily definitive and, together with the above-described effect or instead of the above-described effect, any one of the effects described in the description or another effect that can be identified from the description may be achieved.

### BRIEF DESCRIPTION OF DRAWINGS

FIG. 1 is a diagram illustrating a specific example of a dimensional compression process.

FIG. 2 is a diagram for explaining that the dimensional compression process is performed until an optimum number of dimensions is reached.

FIG. 3 is a diagram illustrating a specific example of condition information.

FIG. 4 is a diagram illustrating a specific example of a physical code in the condition information.

FIG. 5 is a diagram illustrating a specific example of an environment code in the condition information.

FIG. 6 is a diagram illustrating a specific example of the condition information.

FIG. 7 is a diagram illustrating a specific example of the condition information.

FIG. 8 is a diagram illustrating a specific example of accompanying information.

FIG. 9 is a diagram illustrating a specific example of a flow of generating condition information and accompanying information.

FIG. 10 is a diagram illustrating the specific example of the flow of generating condition information and accompanying information.

FIG. 11 is a diagram illustrating a specific example of chronological analysis.

FIG. 12 is a diagram for explaining an example of chronological analysis on a transition of condition.

FIG. 13 is a diagram for explaining an overview of a blockchain that is one type of P2P database.

FIG. 14 is a diagram for explaining the overview of the blockchain that is one type of P2P database.

FIG. 15 is a diagram for explaining the overview of the blockchain that is one type of P2P database.

FIG. 16 is a diagram illustrating an example of a configuration of a biological information processing system according to the embodiment.

FIG. 17 is a block diagram illustrating an example of a functional configuration of an application backend 100.

FIG. 18 is a block diagram illustrating an example of a functional configuration of an internal server 200.

FIG. 19 is a diagram illustrating a specific example of transaction data that is generated by a transaction generator 212.

FIG. 20 is a flowchart illustrating a specific example of a whole process of registering condition information in a P2P database.

FIG. 21 is a flowchart illustrating a specific example of a process of determining whether registering condition information in a P2P database is appropriate.

FIG. 22 is a flowchart illustrating a specific example of a process of generating condition information and accompanying information.

FIG. 23 is a flowchart illustrating a specific example of a process of making a proposal based on condition information.

FIG. 24 is a flowchart for explaining a specific example of a case of use of the biological information processing system according to the embodiment.

FIG. 25 is a block diagram illustrating an example of a hardware configuration of an information processing apparatus 900 that embodies an application backend 100 or the internal server 200 (or an external server 300).

### DESCRIPTION OF EMBODIMENTS

With reference to the accompanying drawings, preferable embodiments of the disclosure will be described in detail below. In the description and drawings, components that have substantially the same functional configuration are denoted with the same number and thus redundant description thereof is omitted.

Description will be given in the following order.

1. Background
1.1. Linkage of Medical Data between Hospitals
1.2. Individual Medicine
1.3. Use of Blockchain

2. Embodiment
2.1. Condition Allocation
2.2. Chronological Analysis on Condition Information
2.3. P2P Database
2.4. Example of System Configuration
2.5. Example of Functional Configuration of each Device
2.6. Example of Flow of Process of each Device
2.7. Case of Use
2.8. Example of Hardware Configuration of each Device

3. Summary

### 1. BACKGROUND

First of all, the background of the disclosure will be described.

1.1. Linkage of Medical Data Between Hospitals

As described above, linkage of medical data between each hospital has not sufficiently progressed. Various reasons are considered for insufficient progress of medical data linkage and, as one of the reasons, differences in the electronic health record system among each hospital or each doctor are taken. More specifically, items in an electronic health record, the order of the items, and the data form, etc., differ depending on each hospital (or each electronic heath record system that each hospital employs). Particularly, as for items in which entries can be made freely in an electronic health record, the content of data entered in the electronic health record differs depending on each doctor. For this reason, data difficult to understand properly and unnecessary data may be entered in the electronic health record.

Thus, even when an electronic health record is linked from another hospital, each hospital has difficulties in properly utilizing the medical data that is recorded in the electronic health record. For example, each hospital is sometimes required to, for each hospital that links an electronic health record, build a system (for example, a conversion system) to convert medical data that is linked from another hospital into a data form in which the medical data can be utilized and this causes a considerable work. When the medical data that is linked from another hospital contains data difficult to understand properly and unnecessary data, it is required to examine methods for the respective sets of data.

Even when medical data is linked properly between each hospital, because a appropriate analysis method and a utilization method for the linked medical data (for example, a method of analyzing medical data and utilizing the result of analysis for treatment) have not been established and the effect of linking medical data has not been clear, motivation of each hospital in linking medical data has not been high.

Medical data may contain data that can specify an individual and, from the point of view of protection of personal information, it has been difficult to link medical data between hospitals and it has been required to have a consensus of a patient on linking medical data. Determining whether each set of medical data is data that can specify an individual and managing such sets of data distinctively also cause a considerable work. Particularly, as for data that can specify an individual, it is required to manage the data in a secured environment in order to prevent the data from being falsified and misused, and it is not easy to realize a system (system used to link medical data) whose scale is large to be accessible by a lot of hospitals and that is secure.

1.2. Individual Medicine

Considering conventional utilization of medical data, randomized controlled trials (RCT) have been actively performed as a method of evaluating effectiveness of treatment. This is an approach of, in order to prevent occurrence of bias of subjective or arbitrary evaluation, extracting persons randomly from a population, sorting the persons into a treatment group and a control group, and analyzing a result of treatment on each of the persons. Such analysis based on a statistical process enables prediction of the course of treatment on a group (for example, general patients) but there is a limitation in predicting the course of treatment on individual members (for example, individual patients) who belong to the group.

Taking atopic skin as an example, a more specific description will be given. Randomized controlled trials enable development of effective medical treatment against atopic skin and this can be described as equivalent to development of more effective medical treatment based on an average of feature values of the skin of general patients with atopic skin. The background of the onset, such as sensitiveness of skin and a degree of allergy, however differs depending on the patient and thus, logically, there are not patients whose have completely the same symptom even when they have the same disease name, atopic skin.

The effect of a treatment method on which it is determined that the method is effective through randomized controlled trials differs depending on the patient. For this reason, while realization of individual medicine in which medical treatment is changed according to each patient is expected, it can be described that adjusting the amount of medicine or changing the type of medicine while diagnosing the symptoms of the patient based on experiences of each doctor or limited cases in the past are the limit of current individual medicine.

Individual medicine using genome information has been proposed but information that can be obtained by analyzing genome information using current medical techniques is limited (for example, only information on a tendency (type) of a patient can be obtained). It is considered that it is possible to realize more effective individual medicine by linking information on the body of the patient (for example, genome information, anthropometric information, diagnostic information, treatment information, operation information, or information representing the condition of the body of the patient, such as a blood pressure and an electrocardiogram (ECG), that is acquired using a wearable terminal device that is worn by a patient, which will be referred to as “physical information” below), or information on an environment that affects the patient (for example, information on lifestyle habits of the patient or medication information, or information representing the condition of the environment of the patient, such as an acceleration or an angular velocity that is acquired by a wearable terminal device that is worn by the patient, which will be referred to as “environment information” below) and using the information for analysis; however, it is hard to say that a system for individual medicine using such information has been built. Furthermore, it is hard to say that a system for individual medicine has been built by combining medical information that a medical setting records and non-medical information that each individual records.

Considering from a different point of view, it is argued that, in the current medicine, not definitive therapy to control the root cause of a disease but supportive therapy to increase spontaneous remission to promote cure by administering treatment to reduce main symptoms is often focused on. For example, because definitive therapy against abnormality of immune that is the root cause of atopic skin has not been sufficiently established, supportive therapy to reduce inflammation with steroid topical medication or antihistamine has been widely practiced as medical treatment against atopic skin. It can be described that the current medicine practices diagnosis and treatment based on understanding of features of a patient at the time of practice of diagnosis and treatment, that is, based on spatial features.

It is considered that, in order to promote and develop definitive therapy (or more effectively implement supportive therapy), analyzing not only data that is obtained during diagnosis but also time-series data in which data is arranged chronologically (features over time) is effective. For example, analyzing time-series data in which physical information or environment information on the patient are arranged chronologically clarifies the background of the onset per patient and enables more effective treatment. Currently, however, it is hard to say that a system enabling collection of and analysis on time-series data of physical information or environment information has been built.

1.3. Use of Blockchain

As one of techniques that are expected to link medical data between hospitals and link physical information and environment information in order to realize individual medicine, there is “blockchain”.

A “blockchain” is data in which multiple blocks in which data (transaction data) is stored are chained like a chain using hash values, or the like. Multiple information processing devices (peers and node devices) manages the block chain in a distributed manner, thereby securing authenticity of data that is stored in the blockchain. Furthermore, with the blockchain, it is expected that intervening of approval by the patient in sharing personal information on the patient secures the right of self-determination of the patient.

Basically, the block chain keeps storing data that was registered in the past and thus there is a possibility that the data size of the whole block chain would be enormous as the blockchain is practiced. Data in a large size increases the burden of computing for hashing. For this reason, registering large-sized data in a blockchain is not preferable. Thus, for example, a method of registering only (part of) an electronic heath record in a block chain, a method of registering, in a blockchain, only a site (pass) in a given database in which medical data is saved, etc., have been proposed. In the former method, however, because the electronic health record system differs between hospitals as described above, it is still difficult to link and utilize medical data and it is not possible to link physical information and environment information that are managed outside the electronic health record. The latter method requires access to the given database to acquire medical data and additionally requires a system for control on access to the database and thus it is hard to say that medical data is linked appropriately. From the point of view of protection of personal information, a problem still remains in both the former and latter methods in that it is necessary to have a consensus from the patient on linking the medical data and it is necessary to determine whether each set of data is data that can specify an individual and manage such data distinctively.

### 2. EMBODIMENT

The disclosers reached creation of the technique according to the disclosure in view of the above-described background. A biological information processing apparatus according to the disclosure acquires biological information on a subject (covering a patient), generates condition information representing a biological condition of the subject based on the biological information, and registers the condition information in a P2P database (covering a blockchain). More specifically, the biological information processing apparatus generates a condition code representing a biological condition of the subject by encoding the biological information using a given method and registers condition information containing the condition code in the P2P database. An embodiment of the disclosure will be described in detail below.

2.1. Condition Allocation

First of all, details of a process performed by the biological information processing apparatus according to the embodiment to generate condition information based on biological information (the process is referred to as “condition allocation” below) will be described.

The “biological information” that is used for condition allocation is a concept covering physical information or environment information on the subject.

The “physical information” covered by biological information is information on the body of the subject (covering a patient) as described above and includes, for example, anthropometric information (for example, a height, seating height, a weight, a BMI (Body Mass Index), a body fat percentage, a visual acuity, or an audibility); diagnosis (medical interview) information (for example, a name of disease, an X-ray image, an MRI image, a gamma GTP, or a subjective symptom); treatment information (for example, the content of treatment or the time of treatment); or operation information (for example, the content of an operation or the duration of an operation). Note that the content of physical information is not limited to them. For example, the physical information may contain genetic information, such as genome information or epigenome information; information of molecular indices obtained using liquid biopsy, such as hormone, cytokine, growth factors, and circulating nucleic acids in a body fluid sample, such as the blood; or information representing the physical condition of the patient acquired using a wearable terminal device that is worn by the subject and a sensing terminal device having a function of sensing the subject from radio waves, image information, or the like (for example, information obtained by various sensors that the sensing terminal device includes, such as vital signs including a heart rate, autonomic nerves, and a sleep rhythm, an amount of oxygen in the blood, a blood glucose level, a blood pressure, or uric protein). The physical information may contain attribute information on the subject (covering a patient) that is recorded in the electronic health record (for example, a name, a date of birth, an age, a gender, a blood type, an address, a phone number, or a place of employment) and medical information, such as the names of the doctor and hospital that administered treatment and performed an operation on the subject. The physical information, such as anthropometric information, treatment information, and operational information, may contain elements as a time series (a log of each set of information). Treatment refers to medical practices not corresponding to operations.

The “environment information” contained in the biological information is information on the environment that affects the subject (covering a patient) as described above and, for example, contains information on lifestyle habits of the subject (for example, habits, such as smoking, drinking alcohol, diets, sleeping, or exercise, and stresses), medication information (for example, a type of drug, a dosage and an administration), information representing the condition of the environment of the patient acquired by the wearable terminal device that is worn by the subject or the sensing terminal having a function of sensing the subject from waveforms or image information (for example, information acquired by the various sensors of the sensing terminal, such as an acceleration, an angular velocity, etc.), and information that is obtained using the liquid biopsy, such as molecular indices of blood, body fluids, etc. The environment information may contain information on the environment that is estimated from the physical information (for example, vital signs, such as a heart rate, autonomic nerves, and a sleep rhythm, an amount of oxygen in the blood, a blood glucose level, a blood pressure, or uric protein). For example, the environment information may contain information, such as a time to wake up that is estimated from the sleep rhythm and exercise information) that is estimated from the heart rate. When it is information for estimating an environment of the patient, the physical information may be partly recorded as the environment information. The content of the environment information is not limited to them. The information that is acquired using the various sensors may be sensor information itself or may be feature value information of the sensor information that is output because the sensor information is analyzed (from the point of view of processing efficiency and data size, it is preferable that the information acquired using the various sensors be feature value information of the sensor information). Medication by the doctor during the treatment or operation is preferably incorporated in the treatment information or the operation information and medication by the subject himself/herself (ingestion of drug) is preferably contained in the medication information.

The condition allocation refers to inputting the above-described biological information (containing the physical information or the environment information) to a given classifier, thus outputting a classification result, and, using the classifying result, generating condition information. The classifier may simply refer to a table (more specifically, a table in which biological information and conditions are associated with each other) or an approach of machine learning. For example, condition allocation may be performed in a way that the biological information is converted using a table serving as a classifier as follows “atopic skin Type A→1A, the medical condition site is the right upper arm→7, a lot of redness→3). Condition allocation on the biological information may be performed using a method of machine learning, such as a support vector machine or a neural network. For example, a classifier obtained by performing learning using learning data in which given biological information and conditions are associated is generated and the biological information is input to the classifier and thereby dimensional compression is performed and condition allocation is performed. Condition allocation in which the biological information is input to a neural network with a given parameter and a vector value or a scholar value obtained by performing dimensional compression is performed serves as a condition may be performed. Note that, because classification by the aforementioned classifier enables compression of dimensions of the biological information, the condition allocation can be also referred to as generating condition information by performing a process of compressing the dimensions of the biological information using a table conversion or machine learning approach (referred to as “dimensional compression process” below).

As for the support vector machine, for example, combining multiple support vector machines builds a support vector machine model for multiclass classification and inputting learning data (the biological information) to the model generates a classifier. As for the neural network, building a multilayer neural network, inputting learning data (data of combination of the biological information and conditions corresponding to the biological information), and adjusting the parameter of the multiplayer neural network generate a classifier. The biological information processing apparatus may perform condition allocation using an artificial intelligence (AI) as the classifier. Dimensional compression using machine learning will be described in detail below.

A specific example of the dimensional compression process performed in condition allocation will be described. For example, when blood pressure information on the subject (for example, numeric data of the systolic blood pressure and the diastolic blood pressure) is input to the classifier described above and the classifier accordingly determines that the subject is “Type 1: Optimum blood pressure” from among “Type 1: Optimum blood pressure”, “Type 2: Normal blood pressure” and “Type 3: High blood pressure”, the biological information processing apparatus may output “1”, thereby implementing the dimensional compression process. For example, when acceleration information on the subject (for example, numeric data of acceleration within a given duration) is input to the classifier described above and accordingly it is determined that the lifestyle habits of the subject is “Type 3: Evening person” from among “Type 1: Morning person”, “Type 2: Standard” and “Type 3: Evening person”, the biological information processing apparatus may output “3”, thereby implementing the dimensional compression process. By using more types of biological information, the biological information processing apparatus is able to improve accuracy of condition allocation and increase possible conditions for allocation. For example, by further using not only the blood pressure information and acceleration information but also heart rate information and blood glucose level information, the biological information processing apparatus is able to increase accuracy of condition allocation and the number of possible conditions for allocation.

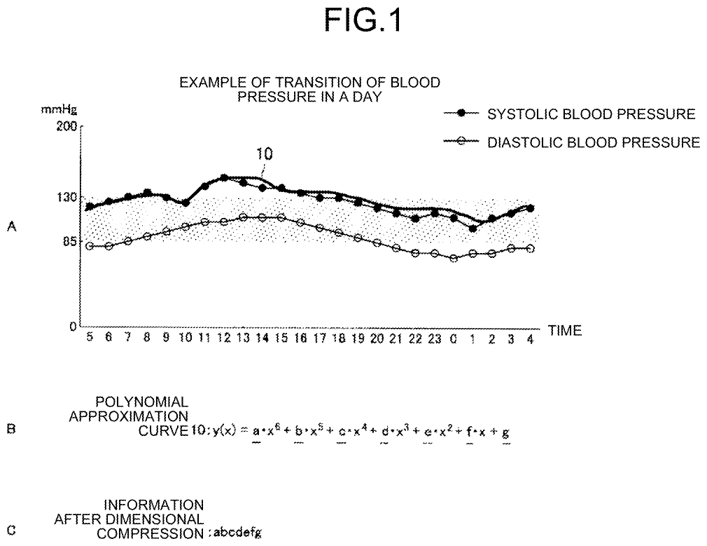

When performing classification using the above-described classifier, the biological information processing apparatus may convert biological information into a form that is easier to classify. For example, the biological information processing apparatus may perform dimensional compression by performing given approximation processing on the biological information to simplify the biological information (output an approximation model), thereby converting the biological information into a form that is easy to classify. For example, as illustrated in FIG. 1, when blood pressure information (in the example illustrated in FIG. 1, information on shifts in blood pressure in a day) is acquired as biological information, as illustrated in FIGS. 1A and 1B, the biological information processing apparatus analyzes the information, thereby calculating a polynomial approximation curve 10 representing shifts in the upper pressure (the systolic blood pressure). As illustrated in FIG. 1C, the biological information processing apparatus may output a data string obtained by connecting each polynomial coefficient, thereby compressing the dimensions of information on the upper blood pressure (systolic blood pressure). Similarly, the biological information processing apparatus may compress the dimensions of information on the lower blood pressure (diastolic blood pressure). The biological information processing apparatus may reduce granularity of the biological information, thereby converting the biological information into a form that is easier to classify. For example, when information on the BMI is acquired as biological information and the information is “17.55” (in other words, information containing up to the hundredths place), the biological information processing apparatus may make a conversion by rounding off after the hundredths place, thereby converting the biological information into a form that is easier to classify.

When performing classification using the above-described classifier, the biological information processing apparatus is able to perform classification using a different classifier depending on the information contained in the biological information. For example, the biological information processing apparatus may classify blood pressure information using the table as a classifier and classify acceleration information using the support vector machine approach as a classifier. The biological information processing apparatus may perform classification using a combination of multiple classifiers. For example, after classifying blood pressure information using the table as a classifier, the biological information processing apparatus may further perform classification using the support vector machine approach as a classifier.

After performing classification using different classifiers depending on the types of biological information, the biological information processing apparatus may further perform a dimensional compression process on each of the results of classification, thereby performing condition allocation. For example, the biological information processing apparatus generates a matrix {A, B, C, . . . } by arranging character strings representing respective results of classification of multiple types of biological information: a matrix A representing a result of condition allocation of the blood pressure information, a matrix B representing a result of condition allocation of the acceleration information, a matrix C representing a result of condition allocation of the heart rate information, . . . . Based on the matrix, the biological information processing apparatus performs condition allocation by performing the dimensional compression process and generates information representing the condition of the owner of the biological information (condition information). In other words, the condition is represented as a condition vector (matrix). The physical information and environment information are also represented by condition vectors (matrices).

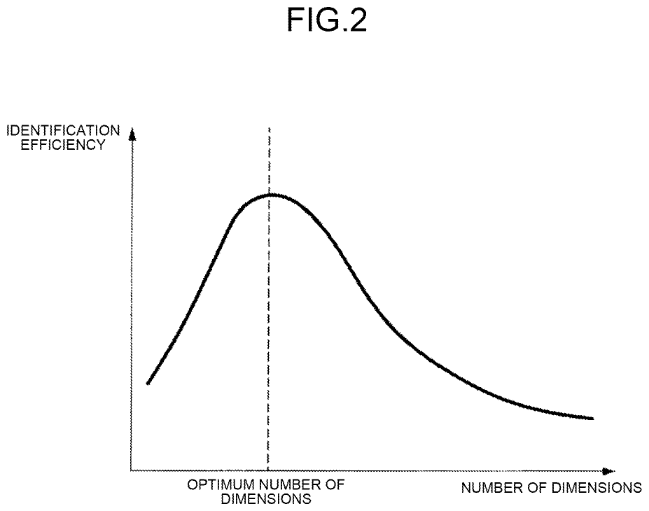

Physical feature values are enormous and thus the biological information processing apparatus preferably performs the process of dimensional compression to dimensions by which the features of the biological condition of the subject can be identified properly (a degree at which the features of the biological condition of the subject can be identified is referred to as “identification efficiency” below). With reference to FIG. 2, the correlation between the number of dimensions of the biological information and the identification efficiency will be described. In general, when the number of dimensions of the biological information is too small, the identification efficiency tends to decrease because the amount of information used to identify features of the biological condition is small. The identification efficiency increases as the number of dimensions of the biological information increases and, after the number of dimensions of the biological information exceeds a given value (“optimum number of dimensions” in FIG. 2), the identification efficiency tends to decrease because a lot of unnecessary information is contained. Thus, it is preferable that the number of dimensions of the dimensional information be reduced to the “optimum number of dimensions” in FIG. 2 (or a number of dimensions close to the “optimum number of dimensions”). The biological information processing apparatus may calculate an optimum number of dimensions through learning by the machine learning approach on the resultant identification data obtained by performing the dimensional compression process on the biological information on a considerable number of subjects. Note the method of calculating an optimum number of dimensions is not limited to this.

The biological information processing apparatus may perform the dimensional compression process based on medical knowledge and perform condition allocation. Each of disorders, such as cancers and atopic skin, is evaluated using eight feature values (immunogram) that are extracted based on medical knowledge from the feature values of enormous biological information. The biological information processing apparatus is able to perform dimensional compression by extracting a feature value associated with each disease that is specified based on medical knowledge from the input biological information. For example, atopic skin is known as being evaluable based on clinical trials using the eight feature values of the biological information representing a cutaneous barrier function, immunoregulation, and bacterial flora. Thus, an input of “atopic skin” that is diagnostic information triggers the biological information processing apparatus to extract the eight feature values on atopic skin from the input biological information, perform the dimensional compression process, thereby generate condition information. After the dimensional compression process based on the medical knowledge, the biological information processing apparatus may further perform dimensional compression using the above-described classifier, thereby generating condition information. The dimensional compression method makes it possible to, for various disorders, perform layering that has clinical meaning medically.

The biological information processing apparatus performs the dimensional compression process using the above-described classifier, thereby generating an encoded physical code or an encoded environment code. The biological information processing apparatus performs the dimensional compression process using a classifier on at least any one of the physical code and the environment code, thereby generating an encoded condition code. The biological information processing apparatus further incorporates the encoded information in the condition information. It is preferable that the dimensional compression process makes the data size of the condition information be equal to or smaller than the block capacity of a block chain.

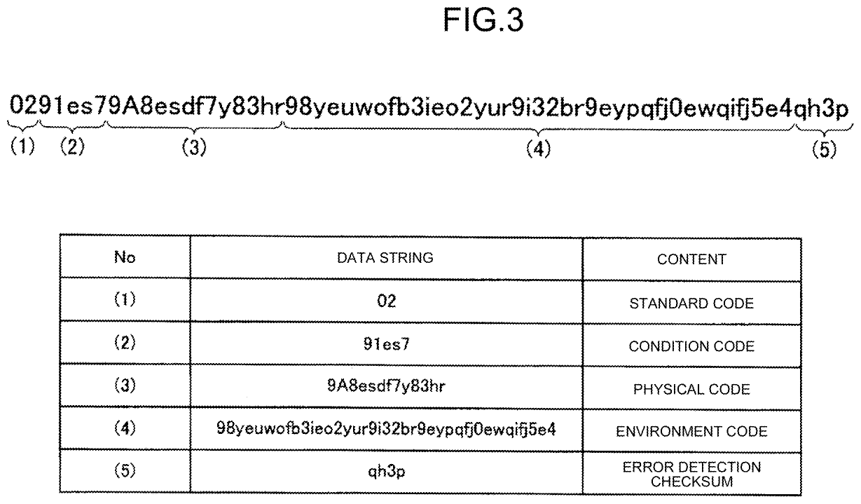

With reference to FIGS. 3 to 8, a specific example of the condition information that is generated by the dimensional compression process on the biological information will be described.

As described above, the “condition information” refers to information representing biological condition of a subject (covering a subject). With reference to FIG. 3, a specific example of the condition information will be described. For example, the condition information includes a standard code, a condition code, a physical code, an environment code, and an error detection checksum. As illustrated in FIG. 3, the condition information is expressed by a character string of 64 digits of numbers and alphabets “0291es79A8esdf7y83hr98yeuwofb3ieo2yur9i32br9eypqfj0ewgifj5 e4qh3p”.

The “standard code” is information representing a method that is used to generate the condition information based on the biological information (note that, in other words, the standard code can be referred to as a method code representing the method used to generate the condition information). In FIG. 3, the standard code corresponds to “02” of Data String No. (1). More specifically, it is provided that the method of generating a physical code, an environment code, and a condition code, which will be described below, based on the biological information is standardized according to given standards (for example, ISO (International Organization for Standardization) standards). In other words, it is provided that subjects, such as multiple companies, are able to use the biological information processing system according to the embodiment to generate condition information using the method that is standardized according to the given standards. Incorporating the standard code in the condition information allows the user of the biological information processing system to specify the method that is used to generate the condition information and thus appropriately use the condition information.

The “physical code” is information that is encoded by performing the dimensional compression process on the physical information. Encoding the physical information by the dimensional compression process makes it more difficult to specify the individual.

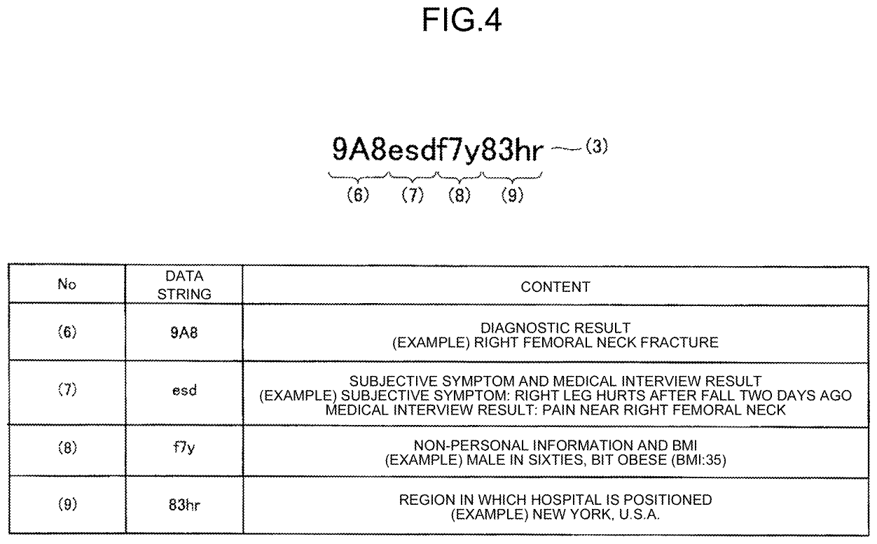

In the example in FIG. 3, the physical code has a data string “9A8esdf7y83 hr” of Data string No (3) and a specific example of the information contained in the data string will be described with reference to FIG. 4. More specifically, as illustrated in FIG. 4, the physical code has a diagnostic result, a subjective symptom and a medical interview result, non-personal information and a BMI, and a region in which a hospital is positioned. Each of the sets of information is encoded by the dimensional compression process and, for example, the diagnostic result is a data string “9A8” representing “a right femoral neck fracture”, the subjective symptom and the medical interview result are a data string “esd” representing “the subjective symptom: the right leg hurts after a fall two days ago, and the medical interview result: a pain near the right femoral neck”, the non-personal information and the BMI are a data string “f7y” representing “a man in sixties, bit obese (BMI: 35)”, and the region in which the hospital is positioned is a data string “83 hr” representing “New York, U.S.A”. Note that the information contained in the physical code is not limited to the example in FIG. 4. The non-personal information is information obtained by processing the personal information such that the individual is not specified.

The “environment code” in FIG. 3 is information that is encoded by performing the dimensional compression process on the environment information. Encoding the environment information by the dimensional compression process make it difficult to specify the individual.

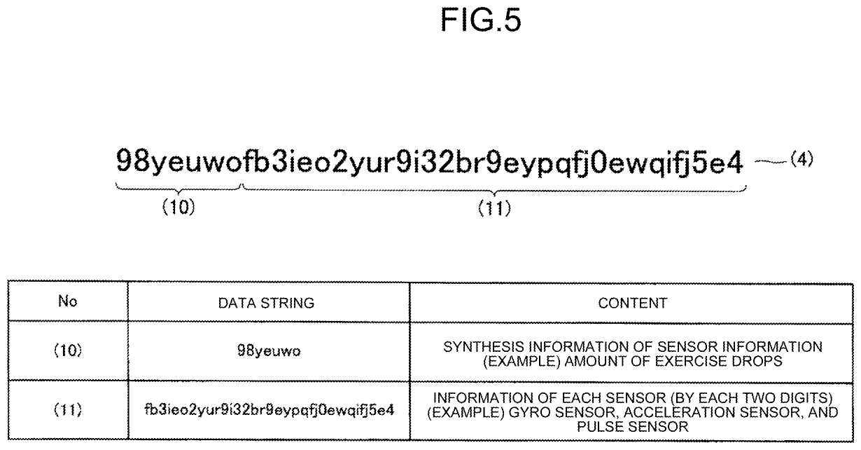

In the example in FIG. 3, the environment code has a data string “98yeuwofb3ieo2yur9i32br9eypqfj0ewqifj5e4” of Data string No (4) and a specific example of information contained in the data string will be described with reference to FIG. 5. More specifically, as illustrated in FIG. 5, the environment code includes synthesis information of the sensor information and information of each sensor. The synthesis information of the sensor information is information that is encoded by performing the dimensional compression process on the information of each sensor and is a data string “98yeuwo” representing “the amount of exercise drops”. The information of each sensor contains information that is encoded by performing the dimensional compression process on the information that is acquired from, for example, a gyro sensor, an acceleration sensor or a pulse sensor. Note that the information contained in the environment code is not limited to the example in FIG. 5. For example, the type of the sensor information contained in the environment code is not particularly limited. In the example in FIG. 5, the information of each sensor is dimensionally compressed to information of two digits per sensor; however, the number of digits of information of each sensor is not particularly limited.

The “condition code” in FIG. 3 is information that represents the biological condition of the subject and that is generated by encoding at least any one of the physical code and the environment code by the dimensional compression process. In FIG. 3, the condition code corresponds to “91es7” in Data string No (2). As in the case of the physical code and the environment code, encoding the condition code by the dimensional compression process makes it more difficult to specify the individual.

The “error detection checksum” is information that is used to detect an error in the condition information (for example, falsification of the condition information or corruption of the condition information due to some kind of cause). More specifically, the biological information processing apparatus that has acquired the condition information performs a given operation (for example, calculation of a hash value) on part of the condition information (more specifically, the part of the condition information excluding the error detection checksum) and confirms that no error is contained in the condition information based on matching between the result of operation and the error detection checksum. Incorporating the error detection checksum in the condition information prevents incorrect condition information from being used for processing.

For example, when an approach of machine learning, such as a support vector machine or a neural network, is used for the dimensional compression process, the biological information is converted into a position in a vector space. Thus, as illustrated in FIG. 5, the condition code, the physical code, and the environment code may indicate positions in the vector space.

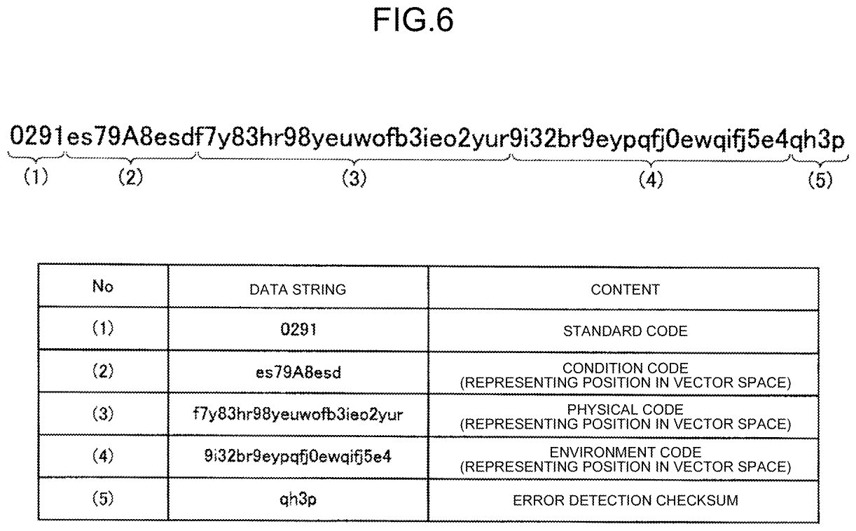

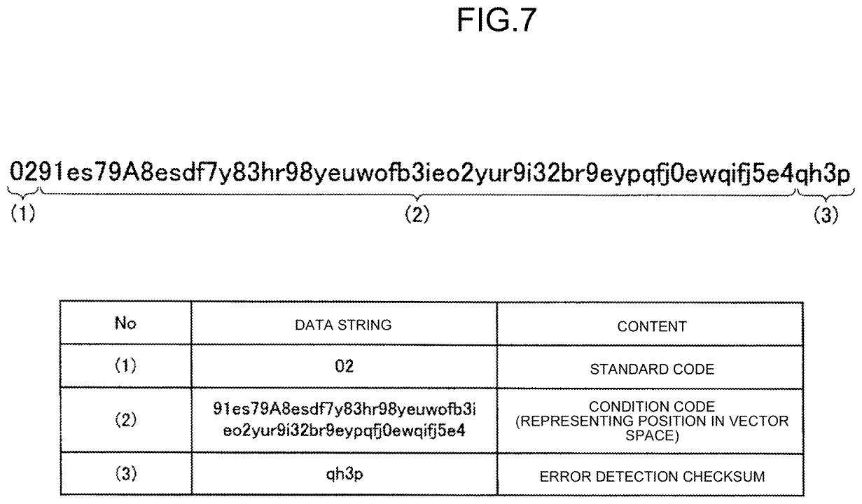

The condition information is not limited to the example illustrated in FIG. 6. For example, the condition information need not necessarily contain all the information illustrated in FIGS. 3 and 6 and the information can be appropriately omitted. Information other than the information illustrated in FIGS. 3 and 6 may be incorporated in the condition information. More specifically, as illustrated in FIG. 7, the physical code and the environment code may be omitted. In FIGS. 3, 6, and 7, from the point of view of process efficiency, etc., the case where the condition information is formed of 64 alphabets and numbers has been described as an example; however, the data length of the condition information is not particularly limited. In the embodiment, the condition information is described as a character string that is expressed using 35 types of characters of 26 types of alphabets and nine types of numbers; however, the method of expressing a character string is not limited. For example, the condition information may be displayed as a hexadecimal data string and may be expressed as “0201B60AC1E5751957A41AB6BEC66E290AF5263E2AD9F9CC9D824BD40A FE0FAF”. The hexadecimal data string has an effect that the storage can be reduced.

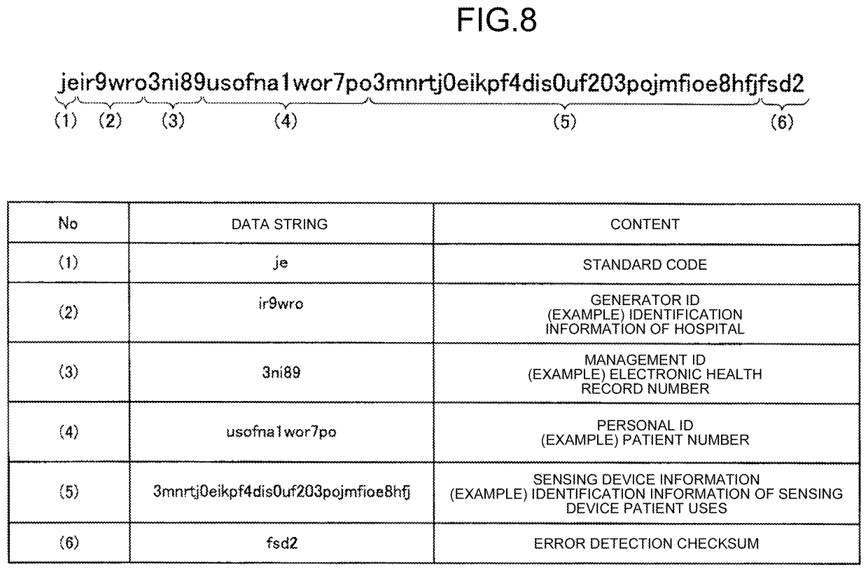

When generating condition information based on biological information, the biological information processing apparatus outputs accompanying information together. The “accompanying information” refers to information that is generated based on information that is not used to generate the condition information among the biological information. With reference to FIG. 8, a specific example of the accompanying information will be described.

As illustrated in FIG. 8, for example, the accompanying information includes a standard code, a generator ID, a management ID, an personal ID, sensing device information, and an error detection checksum.

Like the standard code of the condition information described above, the “standard code” is information representing a method that is used to generate the accompanying information based on the biological information. In FIG. 8, “je” in Data string No (1) corresponds. The standard code may, for example, represent the type of a given conversion process that is used for generation when the accompanying information is generated using the given conversion process (for example, an encryption process or a hashing process).

The “generator ID” is information representing a generator that has generated the condition information and the accompanying information are generated. For example, the generator ID is a data string “ir9wro” of Data string No (2) in FIG. 8 representing “hospital identification information” and is information that is generated by performing the given conversion process (for example, a coding process or a hashing process) on the dentification information (before conversion) that is set previously in each hospital. As described below, the accompanying information is registered in the P2P database together with the condition information and incorporating the generator ID in the accompanying information makes it possible to specify the generator of the condition information and the accompanying information.

The “management ID” is information that is used to manage the condition information and the accompanying information. For example, the generator ID is a data string “3ni89” of Data string number (3) representing an “electronic health record number” and is information that is generated by performing the given conversion process (for example, a coding process or a hashing process) on the electronic health record number (before conversion) that is set in the electronic health record that is used to generate the condition information and the accompanying information. Incorporating the management ID in the accompanying information makes it possible to specify the electronic health record that is used to generate the condition information and the accompanying information, etc.

The “personal ID” is information representing the subject of the condition information and the accompanying information. For example, the personal ID is a data string “usofna1wor7po” of Data string number (4) representing a “patient number” and is information that is generated by performing the given conversion process (for example, a coding process or a hashing process) on the patient number (before conversion) that is set in the patient who is the subject of the condition information and the accompanying information. Incorporating the personal ID in the accompanying information allows the setting that generates the condition information and the accompanying information to specify the subject of the condition information and the accompanying information.

The “sensing device information” is information representing sensing device information that is used to generate the condition information (or the type of the sensing device). For example, the sensing device information is a data string “3mnrtj0eikpf4dis0uf203pojmfioe8hfj” of Data string number (5) representing “identification information of the sensing device that is used by the patient” and is information that is generated by performing the given conversion process (for example, a coding process or a hashing process) on the identification information (before conversion) that is set previously in the sensing device that is used by the patient. Incorporating the sensing device information in the accompanying information, for example, allows analysis on the information of each sensor contained in the condition information.

Like the error detection checksum of the above-described condition information, the “error detection checksum” is information that is used to detect an error in the accompanying information (for example, falsification of the accompanying information or corruption of the accompanying information due to some kind of cause). Incorporating the error detection checksum in the accompanying information prevents incorrect accompanying information from being used for processing.

The accompanying information is not limited to the example illustrated in FIG. 8. For example, the accompanying information need not necessarily contain all the information illustrated in FIG. 8 and the information can be appropriately omitted. Information other than the information illustrated in FIG. 8 may be incorporated in the accompanying information. Like the condition information, the data length of the accompanying information is not particularly limited. The accompanying information is not limited to a single character string. For example, the accompanying information may contain multiple character strings. Alternatively, the accompanying information may be expressed by a hexadecimal character string.

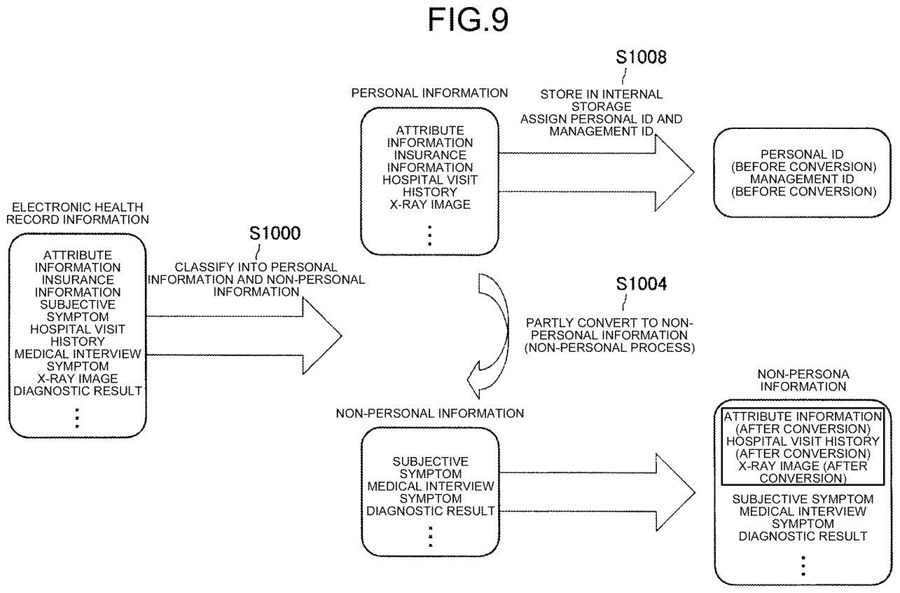

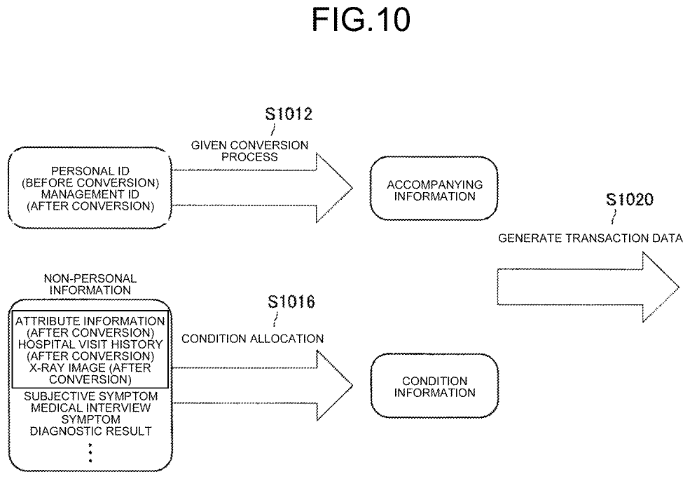

With reference to FIGS. 9 and 10, an overview of a flow of generating condition information and accompanying information described above will be described. FIGS. 9 and 10 illustrate the overview of the process flow of generating condition information and accompanying information using information on an electronic health record that is contained in physical information (denoted by “electronic health record information” in FIGS. 9 and 10). Needless to say, condition information and accompanying information may be generated based on not only physical information but also environment information.

First of all, at step S1000, the biological information processing apparatus classifies electronic health record information into personal information and non-personal information. The “personal information” is information only by which the subject can be specified (or information by which the subject is highly likely to be specified) and contains, for example, attribute information on the subject, insurance information, a hospital visit history, or an X-ray image. The “non-personal information” is information only by which the subject cannot be specified (information by which the subject is not likely to be specified) and contains, for example, a subjective symptom, a medical interview symptom, or a diagnostic result.

At step S1004, the biological information processing apparatus converts part of the personal information into non-personal information (“non-personal process” below). The “non-personal process” refers to converting information into information by which the subject is less likely to be specified by, for example, converting information “Age: 25” contained in the attribute information into information “Age group: twenties” or converting information “Place of employment: ABC Inc.” into information “Occupation: Company Employee”. The non-personal information is converted into a condition code by the following process and, because information, such as the aforementioned age group and occupation, can also be a factor affecting the biological condition of the subject, the biological information processing apparatus adds such information to the non-personal information by performing the non-personal process. The non-personal process may be performed on any information as long as the information can be a factor that affects the biological condition of the subject.

At step S1008, the biological information processing apparatus stores the personal information in a storage in the hospital and issues a personal ID (for example, a patient number) and a management ID (for example, an electronic health record number).

At step S1012, the biological information processing apparatus generates a personal ID (after conversion) and a management ID (after conversion) by performing a given conversion process (for example, a coding process or a hashing process) on a personal ID (before conversion) and a management ID (before conversion) and generates accompanying information containing the personal ID (after conversion) and the management ID (after conversion) (note that description of the conversion process on the generator ID and the sensing device information illustrated in FIG. 8 is omitted.) When performing the hashing process on the personal ID and the management ID, in preparation for the case where a hash collision phenomenon that the same hash values are generated from different character strings, it is preferable that the biological information processing apparatus perform the hashing process on each of the personal ID and the management ID individually and incorporate each of the hash values in the accompanying information. For example, because the personal IDs are saved in association with management IDs in a storage in a hospital (personal IDs are on electronic health records), even when the hash collision phenomenon occurs between personal IDs or management IDs, referring to the records in the storage in the hospital can inhibit confusion of information due to the hash collision phenomenon. It is more preferable that the biological information processing apparatus perform the hashing process with a bit length sufficient to reduce the possibility that the hash collision phenomenon will occur.

At step S1016, the biological information processing apparatus generates condition information by performing condition allocation on the non-personal information by the dimensional compression process. Through the above-described process, condition information and accompanying information are generated. Thereafter, at step S1020, the biological information processing apparats performs a process of registering the condition information and the accompanying information in the P2P database, where transaction data is generated using the condition information and the accompanying information. A specific example of the process of registration in the P2P database will be described below.

2.2. Chronological Analysis on Condition Information

The condition allocation performed by the biological information processing apparatus according to the embodiment has been described above. Subsequently, chronological analysis on the condition information will be described.

When registering the condition information that is generated as described above (and the accompanying information) in the P2P database, the biological information processing apparatus determines whether the condition information is effective and, when it is determined that the condition information is effective, registers the condition information in the P2P database.

The biological condition of the subject is expressed by consecution of changes that are irreversible and occurring in the living body (referred to as “irreversible changes” below). For example, a human undergoes irreversible changes from the moment of fertilization to the birth, growth, and aging and then dies. The onset of a disorder also develops from the pre-stage of the onset affected by a potential change to the onset, a disorder of a specific physiological function, a loss of the specific physiological function, a physical disorder, and the death. Thus, in order to properly express the biological condition of the subject, it is more important to identify the irreversible changes.

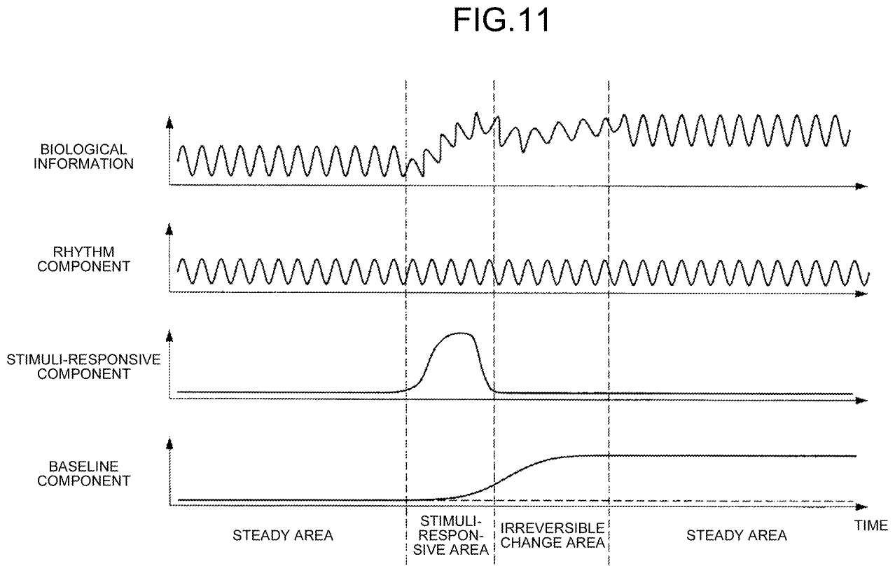

The biological information (the physical information and environment information) from which the condition information originates is information containing changes other than irreversible changes. More specifically, as illustrated in FIG. 11, the biological information (for example, information obtained by various sensors that the sensing terminal device includes, such as vital signs including a heart rate, autonomic nerves, and a sleep rhythm, an amount of oxygen in the blood, a blood glucose level, a blood pressure, or uric protein, and information of molecular indices obtained by liquid biopsy, such as proteins and circulating nucleic acids in a body fluid sample, such as the blood) can be dissolved into a rhythm component, a stimuli-responsive component, and a baseline component.

The “rhythm component” is mainly information based on the 24-hour circadian rhythm and is a component that varies in a given rhythm regardless whether an irreversible change occurs.

The “stimuli-responsive component” is information representing a direct output to an input (stimulus) (response to a stimulus) when the input is made to a living body. For example, when an input that is medication is made to a living body, an effect of the drug appears as a stimuli-responsive component.

The “baseline component” is information that is left after the rhythm component and the stimuli-responsive component are removed from the biological information and is information that irreversibly changes because of some kind of an input (stimulus) that is made to the living body or changes over time (the baseline component is information representing irreversible changes in time-series data). In other words, capturing a change in the baseline component makes it possible to capture an irreversible change. The irreversible change in the biological condition is, for example, an irreversible change over time in genome information and epigenome information in a molecular mechanism, in other words, an irreversible change in chromosome because of genetic modification and epigenetic modification. Discretizing the change in the biological condition over time based on the baseline component makes it possible to express the irreversible change.

Patent Literature 2 above discloses a method of dividing time-series data into a rhythm component, a stimuli-responsive component, and a baseline component. More specifically, Patent Literature 2 above discloses a method of dissolving time-series data on expression of molecules produced in a living body into a rhythm component (a periodic component in Patent Literature 2) using a season adjustment model, into a stimuli-responsive component (an environmental stimuli-responsive component in Patent Literature 2) using a multilinear model, and into a baseline component using a polynomial smoothing spline model.

Using the method described in Patent literature 2, or the like, the biological information processing apparatus performs extraction of a baseline component from the time-series data in which biological information is arranged chronologically as one type of the dimensional compression process and, based on variation in the baseline component, controls registration of the condition information on the subject in the P2P database. More specifically, when it is confirmed that the baseline component varies significantly with respect to a given threshold from the time of previous registration of the condition information on the subject in the P2P database, the biological information processing apparatus determines to register the condition information in the P2P database. Accordingly, the biological information processing apparatus is able to register the more effective condition information in the P2P database. In other words, even data is in an enormous amount like genome information or epigenome information, the biological information processing apparatus is able to compress the data volume into an analyzable form by discretization based on changes in the baseline component and register the data in the P2P database.

By performing chronological analysis on the condition information on the subject, the biological information processing apparatus is able to predict a condition of the subject at a certain future time and make an appropriate proposal based on the result of the prediction.

More specific description will be given. First of all, the biological information processing apparatus acquires condition information on a subject from the P2P database. The biological information processing apparatus then analyzes the condition information, thereby recognizing the condition of the subject. The biological information processing apparatus may acquire multiple sets of condition information that are generated within a given period and analyze the sets of condition information, thereby recognizing a pattern of transition of the condition of the subject (referred to as a “condition transition pattern” below) during the period.

Thereafter, the biological information processing apparatus searches the P2P database for another subject (referred to as a “similar subject” below) who had a condition in the past (or a condition transition pattern) similar to the condition (or the condition transition pattern) of the subject (in other words, the biological information processing apparatus compares the condition information on the subject and condition information on another subject (another set of condition information) and thus extracts a similar subject) with each other). It is more preferable that a similar subject with a condition transition pattern similar to that of the subject during a period as long as possible be found. The number of similar subjects is not particularly limited.

When a similar subject can be found, the biological information processing apparatus acquires condition information on the similar subject at and after the time when the similar subject was similar to the subject in condition (or condition transition pattern) from the P2P database and analyzes the condition information, thereby recognizing the following transition pattern of the similar subject. Accordingly, the biological information processing apparatus is able to predict a condition of the subject at a certain future time.

The biological information processing apparatus is able to notify the subject of the result of prediction of a condition of the subject at a certain future time. It is preferable that the biological information processing apparatus convert the meaning of the condition such that the subject can understand. More specific description will be given. The biological condition of the subject is converted into the condition code, and the subject is unable to recognize his/her biological condition by recognizing only the condition code. The biological information processing apparatus thus converts the condition code such that the subject can recognize the biological condition. For example, the biological information processing apparatus performs inverse conversion the condition code using a given table or verbalize the condition represented by a position in a vector space (for example, when the position of the subject in the vector space is close to positions of many patients with back problems, the biological information processing apparatus notifies that the subject in a condition with a back problem).

When it is predicted that the future condition of the subject is not preferable, the biological information processing apparatus is able to represent a method for making the condition of the subject preferable based on the condition transition pattern of the similar subject. More specifically, when there is a condition positioned in a vector space where the condition is considered as unhealthy among conditions in which the subject will be highly likely to be n years later, the biological information processing apparatus makes a comparison with a condition positioned in a vector space where the condition is considered as healthy and proposes a method to be in a healthy condition. For example, the biological information processing apparatus calculates how to update the environment information to achieve a healthy condition and represents information necessary for the update (for example, an amount of exercise and content of medication). When the condition in which the subject will be most likely to be after n years is positioned in a vector space where a condition is considered unhealthy, the biological information processing apparatus may calculate how the condition of the subject will change if given environment information (for example, an amount of exercise and content of medication) is changed and specify and represent environment information for shifting the condition to a vector space where the condition is considered to be healthy. In other words, the biological information processing apparatus may specify a parameter having a point in being changed (a parameter that contributes to shifting the condition to a vector space where the condition is considered as healthy) and present a method of changing the parameter (such as a method that is derived from medical knowledge (for example, prescribing Drug A when the uric acid level is high)). The “vector space where the condition is considered as healthy” is a vector space where conditions of healthy people are assembled.

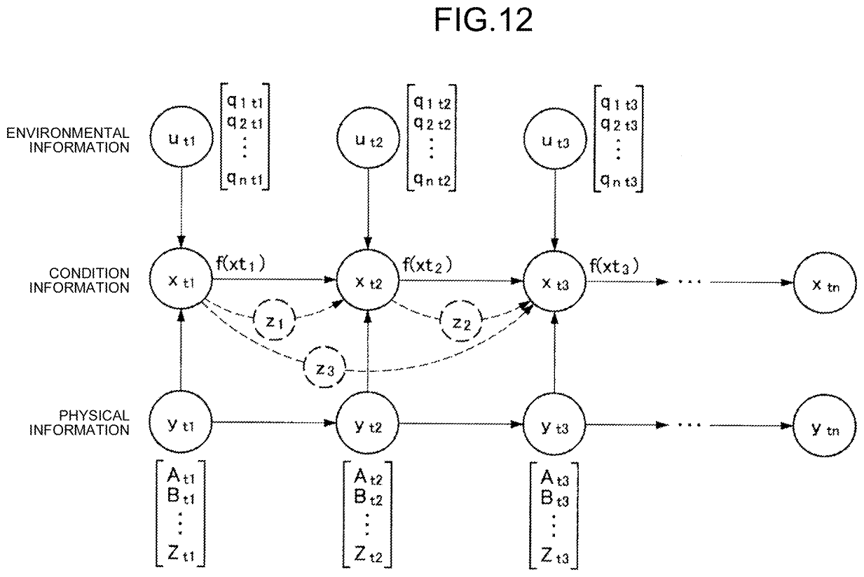

An example of the chronological analysis on the condition transition will be described in detail using FIG. 12. As illustrated in FIG. 12, at a time tn, xtn denotes condition information of a subject, ytn denotes physical information, and utn denotes environment information, where xtn, ytn and utn are expressed by condition vectors (matrices). A condition xtn of the subject is generated based on environment information utn and physical information ytn at that time. For example, when the current time is t3, a condition xt3 of the subject at the current time is calculated from environment information uta and physical information yt3. When a time previous to t3 by 1 chronologically is t2 and a time previous to t3 by 2 chronologically is t1, it can be described that the condition of the subject varies from xt1 to xt2 and xt3 successively.

Furthermore, it can be considered that xtn is a function f(xtn) where the time t is a variable. When it is possible to calculate the function f(xtn), it is possible to estimate a condition xtn of the subject at a future time tn. In the real world, however, parameters are enormous and it is practically impossible to calculate the function f(xtn). Thus, the biological information processing apparatus generates a machine learning model obtained by making a parameter adjustment using changes in condition information (or environment information or physical information) on a subject as learning data and inputs the changes in the condition information on the subject until the current time, thereby statistically estimating a condition of the subject at a future time tn. At a time t1, a probability that a condition xt1 will turn into a condition xt2 at a time t1 is represented as z1 and a probability that the condition xt1 will turn into a condition xt3 is represented by z3. Furthermore, a probability that the condition xt2 will turn into a condition xt3 at a time t2 is represented by z2. Such a condition change model, that is, a model that determines the condition of the current time probabilistically depending on the chronologically previous condition (condition transition probability) is referred to as a hidden Markov model (or a multilayer hidden Markov model). It is more preferable that the above-described estimation of a condition transition probability be performed using an algorithm by which the hidden Markov model is performed easily, for example, a machine learning algorithm using an RNN (Recurrent Neural Network). Not referring to all parameters in the real world but referring to changes probabilistically (statistically) as described above makes it possible to estimate a future condition.

2.3. P2P Database

Chronological analysis on the condition information has been described above. Condition information that is generated by the biological information processing apparatus is registered in the P2P database and is managed. Subsequently, an overview of the P2P database will be described.

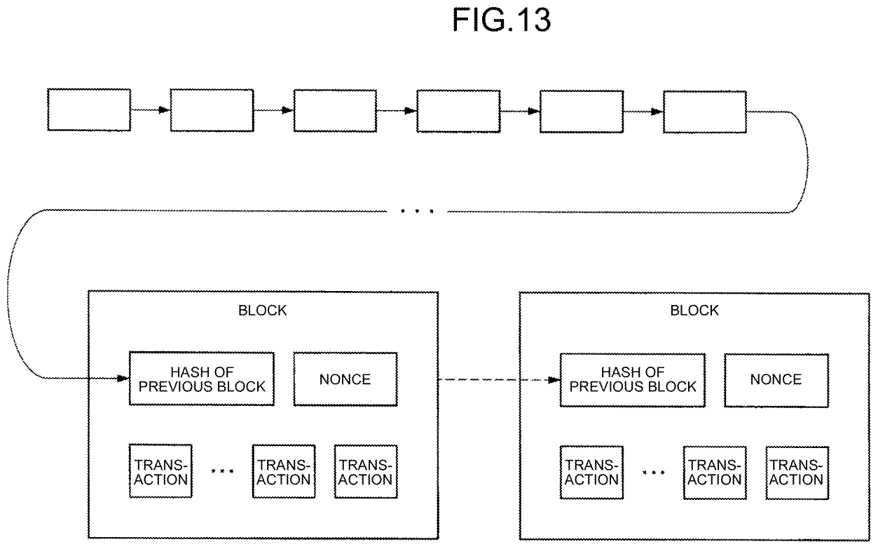

The biological information processing system according to the embodiment uses a distributed P2P database that is distributed in the P2P network. A P2P network is sometimes referred to as a P2P distributed file system. As an example of the P2P database, a blockchain that is distributed in the P2P network is taken. With reference to FIGS. 13 to 15, an overview of a block chain will be descried as an example of the P2P database.

As illustrated in FIG. 13, a block chain is data in which multiple blocks are contained in a chained manner like a chain. In each of the blocks, at least one set of subject data can be stored as transaction data (transaction).

As the block chain, for example, a block chain that is used to trade data of virtual currency, such as Bitcoin, is taken. In a block chain that is used to trade virtual currency data, for example, a hash value of a previous block and a value referred to as nonce are contained. The hash value of the previous block is information that is used to determine whether “it is a correct block” that is continuous correctly from the previous block. A nonce is information that is used to prevent spoofing in authentication using a hash value and using a nonce prevents falsification. As a nonce, for example, a character string, a number string, or data representing a combination thereof is taken.

In a blockchain, assigning a digital signature using an encryption key to each set of transaction data prevents spoofing. Each set of transaction data is open and is shared over the P2P network. Each set of transaction data may be encrypted using an encryption key.

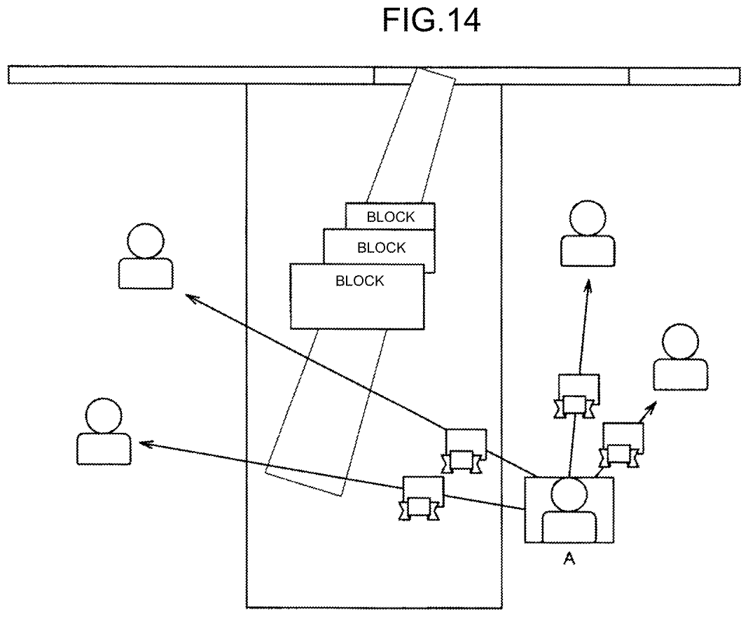

FIG. 14 is a diagram illustrating that subject data is registered by User A in a blockchain system. User A appends a digital signature that is generated using a secret key of User A to subject data to be registered in a blockchain. User A then broadcasts transaction data containing the subject data appended with the digital signature on a P2P network. Accordingly, it is secured that the holder of the subject data (for example, virtual currency) is User A.

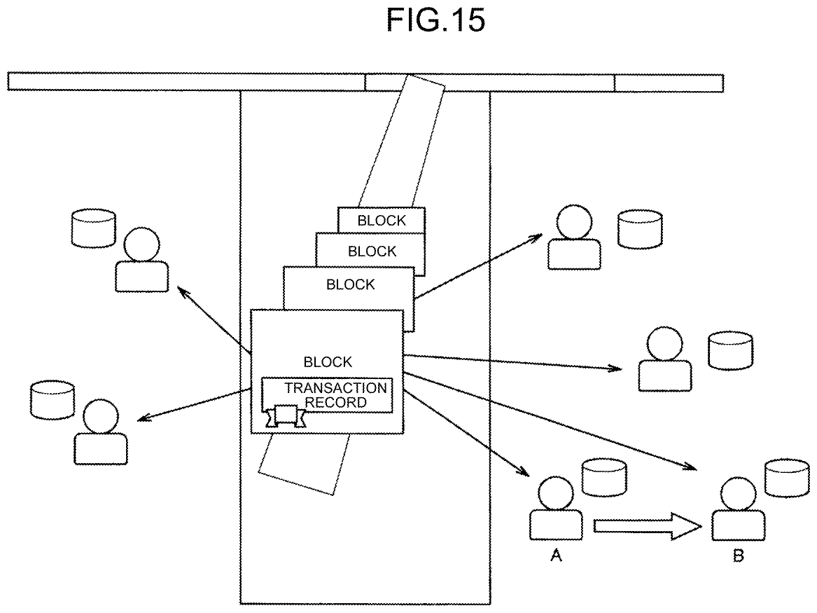

FIG. 15 is a diagram illustrating that the subject data (for example, virtual currency) is migrated from User A to User B in the blockchain system. User A appends a digital signature that is generated using the secret key of User A to transaction data and incorporates a public key of User B in the transaction data. Accordingly, it is represented that the subject data is migrated from User A to User B. In trading subject data, User B may acquire the public key of User A from User A and acquire the subject data that is appended with the digital signature or that is encrypted.

In the blockchain system, for example, using a sidechain technique makes it possible to incorporate other subject data different from virtual currency in an existing blockchain that is used to trade data of virtual currency, such as a Bitcoin blockchain.

As described above, the biological information processing system according to the embodiment uses the distributed P2P database that is distributed in the P2P network; however, note that a distributed network in which a distributed processing is performed by multiple biological information processing apparatuses may be used. The distributed network may be a network including a cloud server that is accessible by, for example, only an authorized user and a biological information processing system in which the above-described condition information is recorded in a storage of the cloud server that is associated with the ID of each user and the condition information is browsed only by an ID that is authorized by each user to access may be built.

2.4. Example of System Configuration

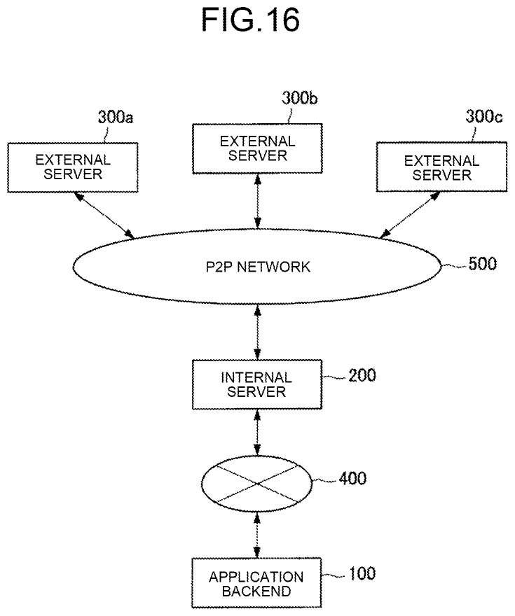

The overview of the P2P database has been described. Subsequently, with reference to FIG. 16, an example of a configuration of the biological information processing system according to the embodiment will be described.

As illustrated in FIG. 16, the biological information processing system according to the embodiment includes an application backend 100, an internal server 200, an external server 300 (external servers 300a to 300c in FIG. 16), an internal network 400, and a P2P network 500.

Application Backend 100

The application backend 100 is a biological information processing apparatus that is mainly used by a doctor who makes a diagnosis on, administers treatment on, or performs an operation on the patient.

More specially, the application backend 100 accesses a storage in the hospital, a storage of the biological information processing apparatus, or an external server (for example, a cloud server) and thus acquires biological information (physical information or environment information). The application backend 100 generates time-series data by arranging the biological information chronologically and extracts a baseline component from the time-series data using the method disclosed in Patent Literature 2 above, or the like.

Based on variation in the baseline component, the application backend 100 then determines whether to register condition information in the P2P database. When it is determined to register condition information in the P2P database, the application backend 100 performs a given conversion process (a dimensional compression process, an encryption process, or a hashing process) on the biological information, thereby generating condition information and accompanying information.

Thereafter, the application backend 100 provides the condition information and the accompanying information to the external server 300. Accordingly, the external server 300 is able to generate transaction data using these sets of information and register the transaction data in the P2P database.

The above-described process content of the application backend 100 is changeable as appropriate. The type of the apparatus that embodies the application backend 100 is not particularly limited. For example, the application backend 100 can be embodied with a freely-selected device covering a PC (Personal Computer), a tablet PC, or a smartphone.

Internal Server 200 and External Server 300

The internal server 200 is a biological information processing apparatus that is connected to the P2P network 500 and that has shared data (covering a P2P database). The internal server 200 generates transaction data using the condition information and the accompanying information that are provided from the application backend 100. The internal server 200 temporarily stores the transaction data in the shared data, thereby sharing the transaction data with the external server 300.

The external server 300 has the same function as that of the internal server 200 and generates transaction data using the condition information and the accompanying information that are generated by an application backend (not illustrated in FIG. 16) that each hospital includes. The external server 300 temporarily stores the transaction data in the shared data, thereby sharing the transaction data with the internal server 200 and other external servers 300.

The internal server 200 and the external server 300 update the P2P database that each device includes while cooperating with each other and thus maintaining consistency (performing the process is referred to as “forming a consensus” below).

The internal server and the external server 300 are capable of performing not only the process of registering transaction data in the P2P database but also a process of acquiring the transaction data from the P2P database.

When the internal server 200 and the external server 300 access the P2P database (in other words, when registration or acquisition of transaction data are performed), the internal server 200 and the external server 300 basically use a given program that is provided in the P2P databased and that is executed in the P2P database (referred to as a “P2P database program” below). Using the P2P database program, for example, realizes various processes including trading a virtual currency, such as Bitcoin, according to given rules. Providing the P2P database program in the P2P database reduces a risk that the program is fraudulently modified.

The P2P database program is a tune code in Hyperledger; however, the P2P database program is not limited thereto. For example, the P2P database program may refer to a smart contract. The internal server 200 and the external server 300 may properly realize access to the P2P database properly using a program other than the P2P database program.

In the embodiment, description will be given, providing that the internal server 200 and the external server 300 have the same function, but the internal server 200 and the external server 300 may have different functions. For example, a device that approves registration of transaction data in the P2P database (for example, an endorsing peer), a device that gives an instruction for registration to each device after approval (for example, an ordering peer), or a device that registers transaction data in the P2P database (for example, a committing peer) may be provided and the internal server 200 and the external server 300 may share and implement the functions of the devices.

The process content of the internal server 200 and the external server 300 describe above can be changed as appropriate. The type of devices that embody the internal server 200 and the external server 300 is not particularly limited. For example, the internal server 200 and the external server 300 are embodied by a freely-selected device covering a general-purpose computer, a PC, a tablet PC or a smartphone.

P2P Network 500

The P2P network 500 is a network in which the P2P database is distributed. As described above, the internal server 200 and the external server 300 are able to form a consensus with another device by connecting to the P2P network 500.

Note that the embodiment provides that the P2P network 500 is a network of a consortium system that is run by multiple organizations; however, the type of the P2P network 500 is not limited thereto. For example, the P2P network 500 may be a network of a private system that is run by only a single origination or a network of a public system that does not particularly restrict participants.

The communication system that is used for the P2P network 500 or the type of line is not particularly limited. For example, the P2P network 500 may be implemented using a dedicated network, such as an IP-VPN (Internet Protocol-Virtual Private Network). The P2P network 500 may be implemented using a public network, such as a telephone network or a satellite network. The P2P network 500 may be implemented using various types of LAN (Local Area Network) and WAN (Wide Area Network) including Ethernet (trademark). The P2P network 500 may be implemented using a wireless communication network, such as Wi-Fi (trademark) or Bluetooth (trademark).

Internal Network 400

The internal network 400 is a network that connects the application backend 100 and the internal server 200. Like the P2P network 500, the communication system that is used for the internal network 400 or the type of line is not particularly limited.

The example of the configuration of the biological information processing system according to the embodiment has been described. Note that the configuration described above with reference to FIG. 16 is an example only and the configuration of the biological information processing system according to the embodiment is not limited to the example. For example, the internal server 200 may include all or part of the function of the application backend 100. For example, software that provides all or part of the application backend 100 may be executed by the internal server 200. Inversely, the application backend 100 may have all or part of the function of the internal server 200. The configuration of the biological information processing system according to the embodiment is flexibly modifiable according to the specification and operation.

2.5. Example of Functional Configuration of Each Device

The example of the functional configuration of the biological information processing system according to the embodiment has been described. Subsequently, an example of a functional configuration of each device will be described.

2.5.1. Example of Functional Configuration of Application Backend 100

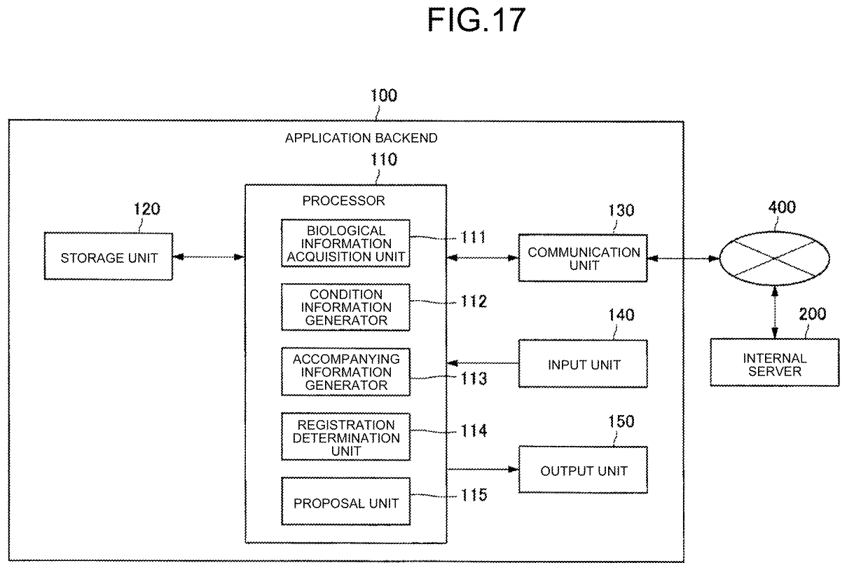

An example of a functional configuration of the application backend 100 will be described. FIG. 17 is a block diagram illustrating the example of the functional configuration of the application backend 100.

As illustrated in FIG. 17, the application backend 100 includes a processor 110, a storage unit 120, a communication unit 130, an input unit 140, and an output unit 150.

Processor 110

The processor 110 is a functional configuration that implements the general process of the application backend 100. For example, an operation input made by a doctor triggers the processor 110 to start a process on registering condition information in the P2P database and, after generating condition information and accompanying information, the processor 110 provides these sets of information to the internal server 200. An operation input made by a doctor triggers the processor 110 to start a process on making a proposal to a patient. The triggers to start these processes are not particularly limited. The content of the processes implemented by the processor 110 is not limited thereto. For example, the processor 110 may implement a process that is generally performed in a PC, a tablet PC or a smartphone (for example, a process performed by an OS (Operating System)). As illustrated in FIG. 17, the processor 110 includes a biological information acquisition unit 111, a condition information generator 112, an accompanying information generator 113, a registration determination unit 114, and a proposal unit 115.

Biological Information Acquisition Unit 111

A biological information acquisition unit 111 is a functional configuration that acquires at least any one of physical information and environment information that is biological information on the subject. As described above, the physical information covers anthropometric information, diagnostic information, treatment information, or operation information and the environment information covers information on lifestyle habits of the subject, medication information, or information acquired by a wearable terminal device that is worn by the subject, and the biological information acquisition unit 111 acquires these sets of information by accessing the storage in the hospital, the storage unit 120 that the application backend 100 includes or an external server (for example, a cloud server). For example, when the biological information is managed using a persona ID, or the like, the biological information acquisition unit 111 searches the storage in the hospital, or the like, for the biological information on the subject using the personal ID, or the like, and acquires the biological information. When the biological information is registered in the P2P database, the biological information acquisition unit 111 may acquiring the biological information by accessing the P2P database via the internal server 200. The biological information acquisition unit 111 provides the acquired biological information to the condition information generator 112, the accompanying information generator 113, and the registration determination unit 114.

Condition Information Generator 112

The condition information generator 112 is a functional configuration that generates condition information representing a biological condition of the subject based on the biological information that is provided from the biological information acquisition unit 111. As described with reference to FIGS. 9 and 10, the condition information generator 112 classifies the biological information into personal information and non-personal information and appropriately performs the non-personal process on part of the personal information (for example, a process of converting information “Age: 25” into “Age group: twenties” is performed). The condition information generator 112 performs condition allocation by performing the dimension compression process on the non-personal information, thereby generating condition information.

Accompanying Information Generator 113

The accompanying information generator 113 is a functional configuration that generates accompanying information when condition information is generated based on the biological information that is provided from the biological information acquisition unit 111. As described with reference to FIGS. 9 and 10, the accompanying information generator 113 classifies the biological information into personal information and non-personal information and stores the personal information in the storage in the hospital, or the like, and issues a personal ID (for example, a patient number) and a management ID (for example, an electronic health record number). When the personal ID and the management ID are already issued, the accompanying information generator 113 acquires these IDs from the issuer. By performing a given conversion process (for example, an encryption process or a hashing process) on the personal ID (before conversion) and the management ID (before conversion), the accompanying information Generator 113 generates a personal ID (after conversion) and a management ID (after conversion) and generates accompanying information containing these IDs.

Registration Determination Unit 114

The registration determination unit 114 is a functional configuration that determines whether to register condition information in the P2P database by determining whether the condition information is effective based on the biological information that is provided from the biological information acquisition unit 111. More specifically, the registration determination unit 114 generates time-series data by chronologically arranging the biological information that is provided from the biological information acquisition unit 111 and, using the method described in Patent Literature 2, or the like, (for example, the polynomial smoothing spline model) extracts the baseline component from the time-series data.

When it is confirmed that the baseline component largely varies with respect to the given threshold from the time of previous registration of the condition information on the subject in the P2P database, the registration determination unit 114 determines to register the condition information in the P2P database. The method of storing the baseline component at the time of previous registration of the condition information on the subject in the P2P database is not particularly limited. For example, when the condition information on the subject is registered in the P2P database previously, the baseline component may be stored in the storage in the hospital or may be registered in the P2P database (when the baseline component is registered in the P2P database, the registration determination unit 114 acquires the baseline component from the P2P database via the internal server 200).

Proposal Unit 115

The proposal unit 115 is a functional configuration that performs chronological analysis on the condition information on the subject, thus predicts a condition of the subject at a future time, and makes a proposal based on the result of the prediction. More specifically, the proposal unit 115 acquires the condition information on the subject from the P2P database via the internal server 200. The proposal unit 115 then recognizes the condition of the subject by analyzing the condition information. The proposal unit 115 may acquire multiple sets of condition information that are generated during a certain period and analyze these sets of condition information, thereby recognizing a condition transition pattern of the subject during the period.

The proposal unit 115 then searches the P2P database for a similar subject who had in the past a condition (or a condition transition pattern) similar to the condition of the subject (or the condition transition pattern). When a similar subject can be found, the proposal unit 115 acquires the condition information on the similar subject at and after the time when the similar subject was similar to the subject in condition (or condition transition pattern) from the P2P database and analyzes the condition information on the similar subject, thereby recognizing the following condition transition pattern of the similar subject. Accordingly, the proposal unit 15 is able to predict a condition of the subject at a future time (n years later). The proposal unit 115 presents the result of the prediction of the condition of the subject n years later to the subject.

When it is predicted that the future condition of the subject is not preferable, the proposal unit 115 is able to present a method of making the condition of the subject preferable based on the condition transition pattern of the similar subject. More specifically, when there is a condition positioned in a vector space where the condition is considered as unhealthy among conditions in which the subject will be highly likely to be n years later, the proposal unit 115 makes a comparison with a condition positioned in a vector space where the condition is considered as healthy, thereby proposing a method to be in a healthy condition. For example, the proposal unit 115 calculates how to update the environment information in order to be in a healthy condition and proposes information necessary for the update (for example, an amount of exercise and content of medication) to the subject. When a condition in which the subject will be most likely to be in n years later is positioned in a vector space where the condition is considered unhealthy, the proposal unit 115 may calculate how the condition of the subject will change if the given environment information (for example, the amount of exercise and the content of medication) is changed, specify environment information for shifting the condition to a vector space where the condition is considered healthy, and represent the environment information to the subject.

The method of making a proposal by the proposal unit 115 is not limited the above-described method. The proposal unit 115 may make a proposal using the given machine learning approach or an artificial intelligence (AI). For example, the proposal unit 115 may adjust the parameter that is used for the process through leaning a considerable number of proposal processes using the above-described approach (for example, generating a classifier formed of a multilayer neural network where the parameter is adjusted using learning data in which transition patterns and proposal content are associated), thereby improving accuracy of proposal.

The method of proposing the content of a proposal to the subject is not particularly limited. For example, the proposal unit 115 may present the content of a proposal to the subject by controlling the output unit 150 and thus displaying the content of the proposal on a display or outputting the content of proposal by sound from a speaker.

Storage Unit 120

The storage unit 120 is a functional configuration that stores various types of information. For example, the storage unit 120 stores information that is used for various processes performed by the processor 110 and information that is generated by the various processes (for example, the biological information covering physical information and environment information, the condition information, the accompanying information, the personal ID, or the management ID). The storage unit 120 stores programs or parameters that are used for the processes performed by the respective functional configurations. The information that is stored in the storage unit 120 is not limited to them.

Communication Unit 130

The communication unit 130 is a functional configuration that communicates with external devices. For example, the communication unit 130 transmits the condition information that is generated by the condition information generator 112, the accompanying information that is generated by the accompanying information generator 113, etc., to the internal server 200 and receives, from the internal server 200, various types of data that are acquired by the internal server 200 from the P2P database. The content of information that the communication unit 130 communicates is not limited to them.

Input Unit 140

The input unit 140 acquires an input made by a doctor. For example, the input unit 140 has an input mechanism, such as a touch panel, a keyboard, a mouse or a button, and, when the doctor performs various operations on the input mechanism, the input unit 140 generates input information based on the operations and provides input information to the processor 110. The input mechanism that the input unit 140 includes and the content of input are not particularly limited.

Output Unit 150

The output unit 150 controls various outputs. For example, the output unit 150 includes an output mechanism, such as a display, a speaker, or a lamp, and displays various types of information on a display according to the result of processes performed by the processor 110 or outputs various types of sound using a speaker. The output mechanism that the output unit 150 includes and the content of output are not particularly limited.

The example of the functional configuration of the application backend 100 has been described. Note that the functional configuration described above using FIG. 17 is an example only and the functional configuration of the application backend 100 is not limited to the example. For example, the application backend 100 need not necessarily have all the configuration illustrated in FIG. 17. The functional configuration of the application backend 100 is flexibly modifiable according to the specification and operation.

2.5.2. Example of Functional Configuration of Internal Server 200

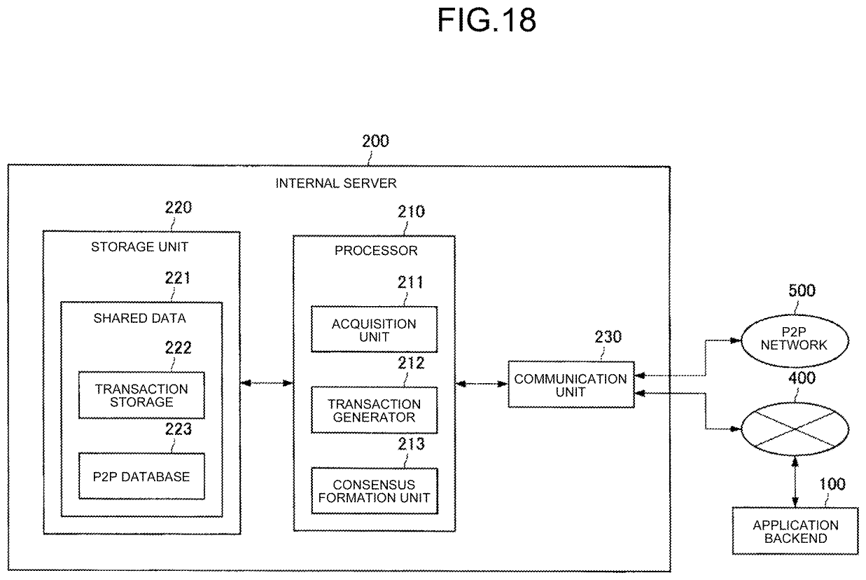

With reference to FIG. 18, an example of a functional configuration of the internal server 200 will be described. FIG. 18 is a block diagram illustrating the example of the functional configuration of the internal server 200.

As illustrated in FIG. 18, the internal server 200 includes a processor 210, a storage 220, and a communication unit 230.

Processor 210

The processor 210 is a functional configuration that implements the general process performed by the internal server 200. For example, the processor 210 controls the start and end of the process of registering the condition information and the accompanying information that are provided from the application backend 100 in the P2P database. The content of the process that is implemented by the processor 210 is not limited thereto. For example, the processor 210 may implement a process that is generally performed by various servers, a PC, a tablet PC or a smartphone (for example, a process performed by an OS). As illustrated in FIG. 18, the processor 210 includes an acquisition unit 211, a transaction generator 212, and a consensus formation unit 213.

Acquisition Unit 211

The acquisition unit 211 is a functional configuration that acquires various types of information. For example, the acquisition unit 211 acquires the condition information, the accompanying information, etc., from the application backend 100 via the communication unit 230. The acquisition unit 211 is also able to acquire various types of information (for example, the condition information on the subject) from the P2P database. Note that the information acquired by the acquisition unit 211 is not limited to them.

Transaction Generator 212

The transaction generator 212 is a functional configuration that generates transaction data to be registered in the P2P database. More specifically, when the condition information and the accompanying information that are provided from the application backend 100 are acquired by the acquisition unit 211, the transaction generator 212 generates transaction data containing these sets of information.

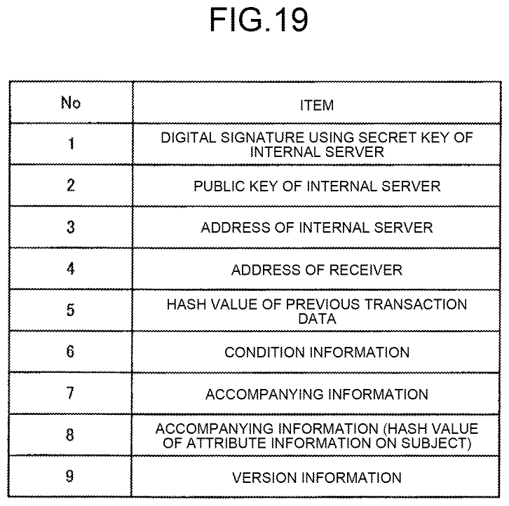

With reference to FIG. 19, a specific example of the transaction data that is generated by the transaction generator 212 will be described. As illustrated in FIG. 19, for example, the transaction data that is generated by the transaction generator 212 includes a digital signature using a secret key of the internal server 200, a public key of the internal server 200, an address of the internal server 200, an address or a receiver, a hash value of previous transaction data, condition information, accompanying information (a hash value of attribute information on the subject), and version information.

“The digital signature using the secret key of the internal server 200” is information that is generated using the secret key of the internal server 200 that generates the transaction data and is information that is used to detect spoofing. “The digital signature using the secret key of the internal server 200” may be replaced with a digital signature that is generated using a secret key that a hospital or a doctor who has generated transaction data other than the internal server 200 holds.

“The public key of the internal server 200” is information that makes it possible to decode the digital signature. Incorporating “the public key of the internal server 200” in the transaction data makes it possible to verify whether spoofing is performed based on the result of decoding the digital signature.

“The address of the internal server 200” is information that can identify the internal server 200 that has generated transaction data. Incorporating “the address of the internal server 200” in the transaction data makes it possible to identify the generator of the transaction data. “The address of the internal server 200” may be replaced with “the public key of the internal server 200” or “the generator ID” illustrated in FIG. 8. “The address of the internal server 200” may be replaced with, for example, an address of a hospital or doctor who has generated transaction data other than the internal server 200.

“The address of the receiver” is information that is registered when there is a receiver that receives the transaction data (or the condition information). Incorporating “the address of the receiver” in the transaction data makes it possible to identify the receiver of the transaction data.

“The hash value of the previous transaction data” is the hash value of the transaction data at the time of previous registration of the condition information on the subject in the P2P database. Incorporating “the hash value of the previous transaction data” in the transaction data represents connection between sets of transaction data on the same subject (in other words, a change in the condition information on the same subject is represented).

“The condition information” is information that has been described with reference to FIG. 3, etc. Incorporating “the condition information” in the transaction data makes it possible to analyze the biological information on the subject.

“The accompanying information” is information that has been described with reference to FIG. 8. Incorporating “the accompanying information” in the transaction data makes it possible to specify the subject of the condition information based on the personal ID and the management ID that are stored in the storage in the hospital, or the like.

“The accompanying information (the hash value of the attribute information on the subject)” is a hash value of the attribute information on the subject that is used to generate the accompanying information (for example, a name, a birth date, an age, gender, a blood type, an address, a phone number, or a place of employment). In order to specify the subject of the condition information based on “the accompanying information”, it is necessary to acquire the personal ID and the management ID that are stored in the storage in the hospital, or the like. On the other hand, incorporating “the accompanying information (the hash value of the attribute information on the subject)” in the transaction data makes it possible to specify the subject of the condition information when the given attribute information can be acquired.

“The version information” is information representing a version of the system (or software) that was used to generate the transaction data (or the condition information). Incorporating “the version information” in the transaction data allows the biological information processing apparatus that has acquired the transaction data to appropriately perform various types of processing using the transaction data.

The content of the transaction data that is generated by the transaction generator 212 is not limited to the above-described content. For example, the transaction generator 212 may generate transaction data by omitting the information described above or adding information that is not described above.

Consensus Formation Unit 213

The consensus formation unit 213 is a functional configuration that performs a process on forming a consensus with the external server 300 (referred to as a “consensus formation process” below) and thus registers the transaction data that is generated by the transaction generator 212 in the P2P database (in other words, the consensus formation unit 213 functions as a register that registers the condition information in the P2P database). The content of the consensus formation process that is performed by the consensus formation unit 213 is not particularly limited. For example, when the P2P database is a blockchain, the consensus formation unit 213 is able to perform the consensus formation process using a consensus algorithm that is known for blockchain techniques. For example, the consensus formation unit 213 performs the consensus formation process using PBFT (Practical Byzantine Fault Tolerance) and thus is able to store transaction data in a new block and register the block in the blockchain. The consensus formation unit 213 may perform the consensus formation process using another consensus algorithm, such as Proof of Work, Proof of Stake, Paxos, Raft, or Sieve.

Storage 220

The storage 220 is a functional configuration that stores various types of information. For example, the storage 220 stores programs or parameters that are used by the respective functional configurations of the internal server 200. The content of information that the storage 220 stores is not limited thereto. As illustrated in FIG. 18, the storage 220 includes shared data 221.

Shared Data 221

The shared data 221 is a set of data that is shared between biological information processing apparatuses that are connected to the P2P network 500. Each of the biological information processing apparatuses acquires the shared data 221 via the P2P network 500 and updates the shared data 221 while maintaining consistency with the shared data that other biological information processing apparatuses store. As illustrated in FIG. 18, the shared data 221 includes a transaction storage 222 and a P2P database 223.

Transaction Storage 222

The transaction storage 222 is a functional configuration that stores transaction data that is not registered in the P2P database 223. The transaction storage 222 registers transaction data that is generated by the transaction generator 212 and transaction data that is generated by the external server 300 and is shared via the P2P network 500. The transaction data that is registered in the transaction storage 222 is basically the same as the transaction data that is registered in the external server 300.

P2P Database 223

The P2P database 223 is a database that is stored in the internal server 200 and is, for example, a blockchain. As described above, transaction data containing the condition information and the accompanying information on the subject is registered in the P2P database 223. The data that is registered in the P2P database 223 is not limited thereto. For example, when transaction data is registered in the P2P database 223 or when a charge is made when the transaction data is acquired from the P2P database 223, data on assets of the subject (for example, coins of Bitcoin) may be registered in the P2P database 223. In the P2P database 223, the above-described P2P database program may be registered. The development language of the P2P database program or the number of P2P database programs that are formed in the P2P database 223 is not particularly limited.

Communication Unit 230

The communication unit 230 is a functional configuration that communicates with external devices. For example, the communication unit 230 receives the condition information and the accompanying information from the application backend 100 or transmits various types of data that are acquired by the acquisition unit 211 from the P2P database 223 to the application backend 100. The communication unit 230 transmits and receives various types of information used for the consensus formation process performed by the consensus formation unit 213 to and from the external server 300. The content of information that the communication unit 230 communicates is not limited to them.

The example of the functional configuration of the internal server 200 has been described. Note that the functional configuration described above using FIG. 18 is an example only and the functional configuration of the internal server 200 is not limited to the example. For example, the internal server 200 need not necessarily include all the configuration illustrated in FIG. 18. The functional configuration of the internal server 200 can be flexibly modified according to the specification and operation.

The external server 300 has the same functional configuration as that of the internal server 200 and thus description thereof will be omitted. Note that the external server 300 need not necessarily have the same functional configuration as that of the internal server 200 and part of the functional configuration may be omitted or the internal server 300 may have a functional configuration that that internal server 200 does not have.

2.6. Example of Flow of Process of Each Device

The example of the functional configuration of each device has been described. Subsequently, an example of a flow of a process of each device will be described.

Process of Registering Condition Information in P2P Database

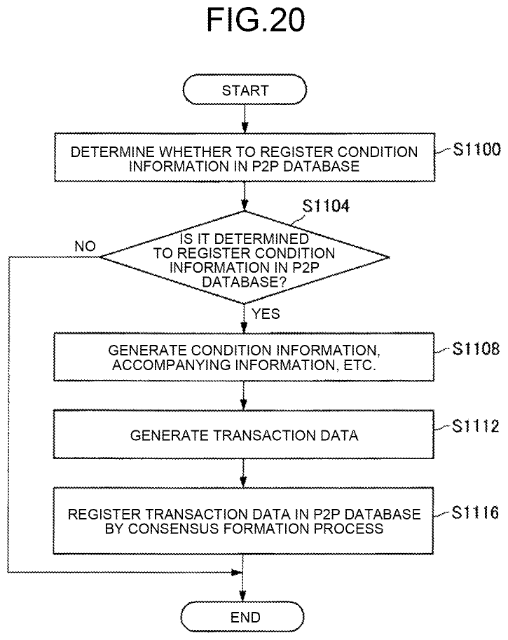

First of all, with reference to FIG. 20, a process of registering condition information in the P2P database 223 will be described. FIG. 20 is a flowchart illustrating a specific example of the general process of registering condition information in the P2P database 223.

At step S1100, the registration determination unit 114 of the application backend 100 determines whether condition information is effective based on biological information, thereby determining whether to register the condition information in the P2P database 223. When the registration determination unit 114 determines to register the condition information in the P2P database 223 (step S1104/Yes), at step S1108, the condition information generator 112 generates condition information on a subject based on biological information and the accompanying information generator 113 generates accompanying information based on the biological information.

At step S1112, the transaction generator 212 of the internal server 200 generates transaction data using the condition information and the accompanying information that are provided from the application backend 100. At step S1116, the consensus formation unit 213 performs the consensus formation process using the consensus algorithm, such as PBFT, thereby registering the transaction data in the P2P database 223 and a series of steps ends.

At step S1104, when the registration determination unit 114 determines not to register the condition information in the P2P database 223 (step S1104/No), the condition information is not registered in the P2P database 223 by the process at steps S1108 to S1116 and a series of steps ends.

2.6.2. Process of Determining Whether Registering Condition Information in P2P Database is Appropriate

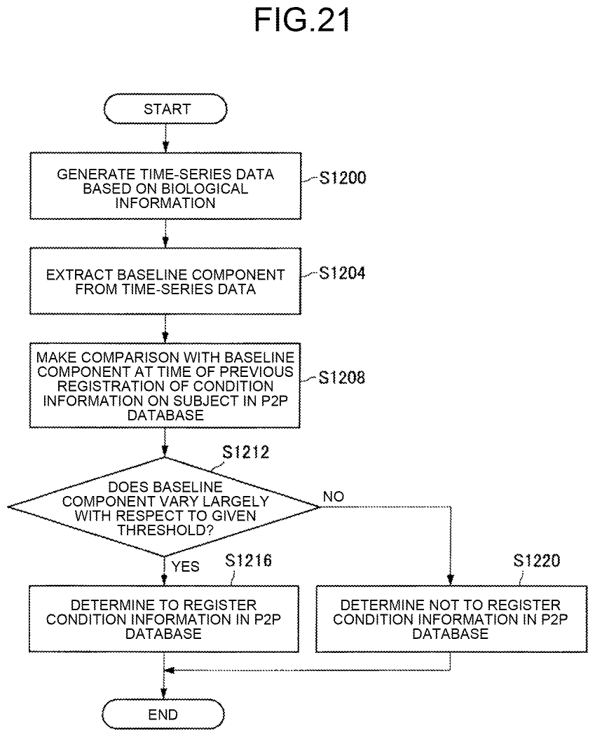

Subsequently, the process of determining whether to register condition information in the P2P database 223, which has been described at step S1100 in FIG. 20, will be described with reference to FIG. 21. FIG. 21 is a flowchart illustrating a specific example of a process of determining whether registering condition information in the P2P database 223 is appropriate.

At step S1200, the registration determination unit 114 of the application backend 100 chronologically arranges the biological information that is provided from the biological information acquisition unit 111, thereby generating time-series data. At step S1204, the registration determination unit 114 extracts a baseline component from the time-series data using the method described in Patent Literature 2 (for example, the polynomial smoothing spline model), or the like.

At step S1208, the registration determination unit 114 compares the baseline component at the time of previous registration of condition information on the subject in the P2P database 223 with the baseline component that is extracted at step S1204. When the baseline component largely varies with respect to the given threshold from the time of previous registration of condition information on the subject in the P2P database (step S1212/Yes), at step S1216, the registration determination unit 114 determines to register the condition information in the P2P database 223 and a series of steps ends. On the other hand, when the baseline component does not largely vary with respect to the given threshold from the time of the previous registration of condition information on the subject in the P2P database (step S1212/No), at step S1220, the registration determination unit 114 determines not to register the condition information in the P2P database 223 and a series of steps ends.

Process of Generating Condition Information and Accompanying Information

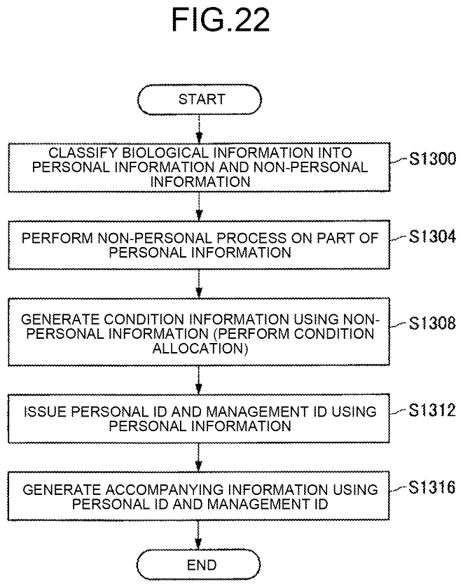

The process of generating condition information and accompanying information that has been described at step S1108 in FIG. 20 will be described. FIG. 22 is a flowchart illustrating a specific example of the process of generating condition information and accompanying information.

At step S1300, the condition information generator 112 and the accompanying information generator 113 of the application backend 100 classify the biological information that is provided from the biological information acquisition unit 111 into personal information and non-personal information. At step S1304, the condition information generator 112 performs the non-personal process on part of the personal information (for example, performs a process of converting information “Age: 25” into information “Age group: twenties”). At step S1308, the condition information generator 112 performs condition allocation on the non-personal information by the dimensional compression process, thereby generating condition information.

At step S1312, the accompanying information generator 113 issues a personal ID (for example, a patient number) and a management ID (for example, an electronic health record number) using the personal information. At step S1316, the accompanying information generator 113 performs the given conversion process (for example, an encryption process or a hashing process) on the personal ID (before conversion) and the management ID (before conversion), thus generates a personal ID (after conversion) and a management ID (after conversion), and then generates accompanying information containing the IDs. Accordingly, a series of steps ends.

2.6.4. Process of Making Proposal Based on Condition Information

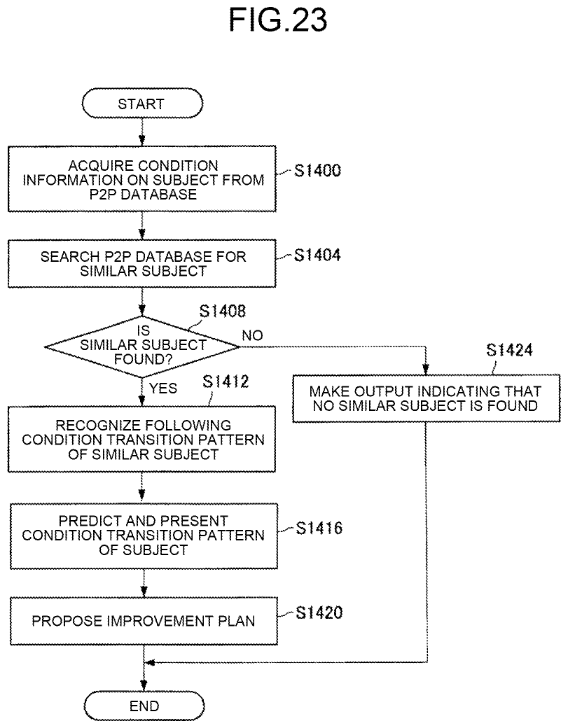

Subsequently, with reference to FIG. 23, the process of making a proposal based on condition information will be described. FIG. 23 is a flowchart illustrating a specific example of the process of making a proposal based on condition information.

At step S1400, the proposal unit 115 of the application backend 100 acquires the condition information on the subject from the P2P database 223 via the internal server 200. At step S1404, the proposal unit 115 analyzes the condition information, thus recognizes the condition of the subject, and then searches the P2P database 223 for a similar subject who had in the past a condition (or a condition transition pattern) similar to the condition (or the condition transition pattern) of the subject.

When a similar subject is found in the P2P database 223 (step S1408/Yes), at step S1412, the proposal unit 115 acquires the condition information on the similar subject at and after the time when the similar subject was similar to the subject in condition (or in condition transition pattern) from the P2P database 223 and analyzes the condition information, thereby recognizing the following condition transition pattern of the similar subject. At step S1416, the proposal unit 115 predicts a condition transition pattern of the subject based on the following condition transition pattern of the similar subject and presents the condition transition pattern to the subject.

At step S1420, the proposal unit 115 proposes an improvement plan to improve the condition of the subject. For example, when there is a condition positioned in a vector space where the condition is considered as unhealthy among conditions in which the subject will be highly likely to be at a certain future time, the proposal unit 115 makes a comparison with a condition positioned in a vector space where the condition is considered as healthy, thereby proposing a method to be in a healthy condition. Accordingly, a series of steps ends.

When no similar subject is found in the P2P database 223 (step S1408/NO), at step S1424, the proposal unit 115 makes an output indicating that no similar subject is found and a series of steps ends.

2.7. Case of Use

The example of the flow of the process of each device has been described. Subsequently, examples of use of the biological information processing system according to the embodiment will be described.

As described above, registering the condition information on the subject in the P2P database allows devices that are able to access to the P2P network 500 to provide various services using the condition information.

It has been described using FIG. 23, etc., that the application backend 100 that a doctor, etc., uses is able to propose an improvement plan to the subject, and the method of utilizing condition information is not limited to this.

For example, a freely-selected company X that is a solution provider (for example, a maker that manufactures products, such as drugs, or a service provider company, such as a training gym) may provide services using the condition information.

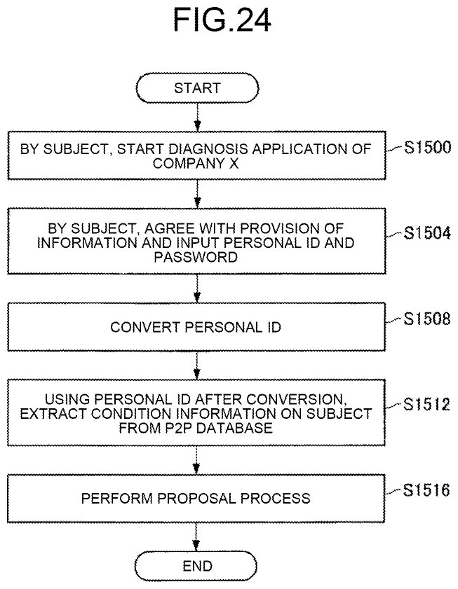

For example, at step S1500 in FIG. 24, a subject operates an electronic device, such as a smartphone, thereby starting a diagnosis application that is run by Company X. At step S1504, the subject makes an input to agree with providing information to the application and inputs a personal ID (before conversion) and a password that are prepared in advance. At step S1508, the application makes a password authentication and, when the authentication succeeds, the application performs a given conversion process (for example, an encryption process or a hashing process) on the personal ID (before conversion), thereby generating a personal ID (after conversion).

At step S1512, the application accesses the P2P network 500 via a given API and extracts condition information on the subject from the P2P database 223 using the personal ID (after conversion). At step S1516, using the condition information, the application performs a proposal process like that described using FIG. 23. For example, when a condition in which the subject will be most likely to be n years later is positioned in a vector space where the condition is considered as unhealthy, the application of Company X calculates how the condition of the subject will change when provision of products from Company X or provision of services of Company X is input as environment information and specifies and proposes a product and the content of service for shifting the condition to a vector space where the condition is considered as healthy.

As described above, the biological information processing system according to the embodiment can be widely used by various providers that provide products and services to subjects.

Furthermore, for example, aggregating condition information that is recorded in the P2P database 223 with respect to each region or age and analyzing the condition information makes it possible to detect a degree of expansion of an infection and a regional disease. For example, condition information that is recorded in the P2P database 223 periodically is acquired and a region in which a hospital contained in a condition code and a physical code is positioned is extracted from the condition information. Furthermore, chronological statistical analysis, for example, analysis using an autoregression model or a moving average model, is performed on the extracted information. Accordingly, it is possible to analyze whether the condition code of each region has a statistical change according to the period, for example, whether the number of people in a high-temperature condition is gradually increasing or there are more people in a high-temperature condition than in other regions. Note that performing analysis per generation makes it possible to further increase accuracy.

Each step in the flowcharts in FIGS. 20 to 24 need not necessarily be processed chronologically in the described order. In other words, each step in the flowcharts may be processed in an order different from the described order or may be processed in parallel.

2.8. Example of Hardware Configuration of Each Device

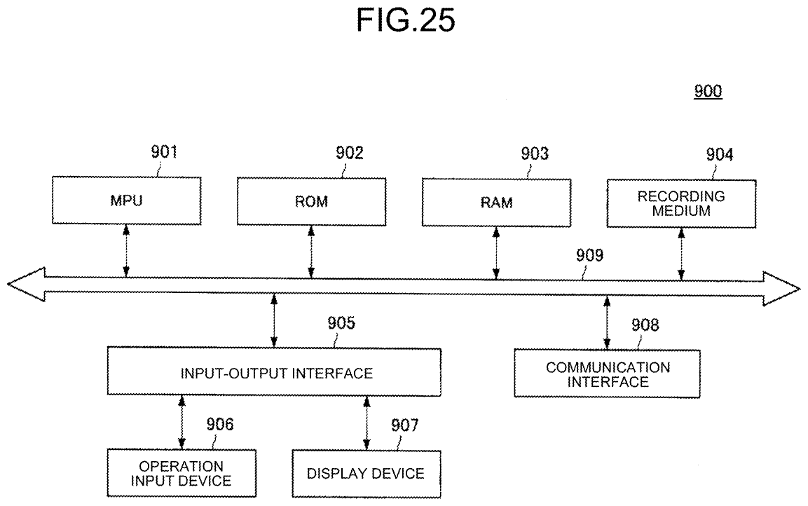

The example of the flow of the process of each device has been described. Subsequently, with reference to FIG. 25, an example of a hardware configuration of each device will be described.

FIG. 25 is a block diagram illustrating an example of a hardware configuration of the application backend 100 or the internal server 200 (or the external server 300). The devices are embodied using an information processing apparatus 900 illustrated in FIG. 25.

The information processing apparatus 900 includes an MPU 901, a ROM 902, a RAM 903, a recording medium 904, an input-output interface 905, an operation input device 906, a display device 907, and a communication interface 908. The information processing apparatus 900, for example, connects each component via a bus 909 serving as a data transmission path.

The MPU 901 is formed of at least one processor that is formed of an operation circuit, such as an MPU, or various processing circuits and the MPU 901 functions as the processor 110 of the application backend 100 or the processor 210 of the internal server 200. Note that these functional configurations may be formed of a dedicated (or general-purpose) circuit (for example, a processor independent of the MPU 901) that can implement the various processes described above.

The ROM 902 stores control data, such as programs and operation parameters that are used by the MPU 901. The RAM 903 temporarily stores, for example, programs that are executed by the MPU 901, etc.

The recording medium 904 functions as the storage unit 120 of the application backend 100 or the storage 220 of the internal server 200 and stores various types of data, such as data on information processing and various programs. As the recording medium 904, for example, a magnetic recording medium, such as a hard disk, or a non-volatile memory, such as a flash memory, is taken. The recording medium 904 may be detachable from the information processing apparatus 900.

The input-output interface 905, for example, connects the operation input device 906 and the display device 907. As the input-output interface 905, for example, a USB (Universal Serial Bus) terminal, a DVI (Digital Visual Interface) terminal, a HDMI (High-Definition Multimedia Interface) (trademark) terminal, or various processing circuits are taken.

The operation input device 906 is, for example, on the information processing apparatus 900 and is connected to the input-output interface 905 in the information processing apparatus 900. As the operation input device 906, for example, a keyboard, a mouse, a keypad, a touch panel, a microphone, an operation button, a rotary selector, such as an orientation key or a jog dial, or a combination thereof is taken. The operation input device 906 functions as the input unit 140 of the application backend 100.

The display device 907 is, for example, on the information processing apparatus 900 and is connected to the input-output interface 905 in the information processing apparatus 900. As the display device 907, for example, a liquid crystal display or an organic EL (electro-luminescence) display is taken. The display device 907 functions as the output unit 150 of the application backend 100.

Needless to say, the input-output interface 905 is connectable to external devices, such as an operation input device or an external display device outside the information processing apparatus 900. The display device 907 may be, for example a device enabling display and user operations, such as a touch panel.

The communication interface 908 is a communication unit that the information processing apparatus 900 includes and the communication interface 908 functions as the communication unit 130 of the application backend 100 or the communication unit 230 of the internal server 200. The communication interface 908 may have a function of communicating with a freely-selected external device, such as a server, in a wired or wireless manner via a freely-selected network (or directly). As the communication interface 908, for example, a communication antenna and an RF (Radio Frequency) circuit (wireless communication), an IEEE802.15.1 port and a transmitter-receiver circuit (wireless communication), an IEEE802.11 port and a transmitter0receiver circuit (wireless communication), or a LAN (Local Area Network) terminal and a transmitter-receiver circuit (wired communication) are taken.

The hardware configuration of the information processing apparatus 900 is not limited to the configuration illustrated in FIG. 25. For example, the information processing apparatus 900 need not include the communication interface 908 when communicating via an external communication device to which the information processing apparatus 900 is connected. The communication interface 908 may be configured as being able to communicate using multiple communication systems. The information processing apparatus 900 need not include the operation input device 906, the display device 907, or the like. All or part of the configuration illustrated in FIG. 25 may be implemented using at least one IC (Integrated Circuit).

Subsequently, dimensional compression using the machine learning approach described above will be described in detail. The case where the dimensional compression process is realized by SAE (Stacked Auto-Encoders) that is one type of multilayer neural network will be described. The SAE is a neural network having a configuration in which neural networks refereed too as auto-encoders are stacked into a multilayer network. In SAE, based on learning data, the parameter of SAE (which means a network coefficient of each layer) is adjusted. The auto-encoder is a neural network having a configuration in which the number of neurons (the number of units) is equal between an input layer and an output layer and the number of neurons in an intermediate layer (also referred to as a hidden layer) is smaller that that of the input layer (or the output layer). Learning of the SAE is performed per auto-encoder forming the SAE and, for example, learning by backpropagation using learning data is performed to adjust the parameter.

For example, a classifier is generated by performing learning using learning data in which biological information and conditions are associated while dimensional compression and dimensional decompression is performed using 49 dimensions, 16 dimensions, three dimensions, 16 dimensions, and 49 dimensions as the numbers of dimensions of the respective layers of the SAE formed of five layers and thus making a parameter adjustment enabling proper classification. When biological information is input to the classifier, compression to three dimensions is performed and the biological information is converted into a value in a three-dimensional space vector. Classification of condition may be also performed. Using the machine learning approach thus makes it possible to generate a value in a freely-selected n-dimensional space vector obtained by dimensional compression or a result of classification. The value in the n-dimensional space vector is dealt with as a condition (or a condition code), or the result of classification is dealt with as a condition (or a condition code). Note that the number of dimensions in dimensional compression is an item to be designed and is not limited to three dimensions if classification is possible and any number of dimensions can be employed. When dimensional compression is possible, the machine learning approach is not particularly limited.

This, for example, makes it is possible to perform table conversion on information “Having atopic skin of Type A, a lot of redness on the right upper arm, BMI of 20, . . . ” to “Type 1 with tendency of causing skin inflammation, severity of reaction of Level 2, BMI of 5 representing an average, . . . ”, thereby generating a sequence vector X (1, 2, 2, . . . ). By inputting the sequence vector X into the neural network on which a parameter adjustment is made previously using learning data containing a given condition and a sequence vector, it is possible to generate a condition Y. Note that the condition Y may be a value in a n-dimensional space or may be a result of classification along a table that is prepared in advance.

For example, by inputting biological information as a1=height and a2=weight in each item, such as BMI, a matrix {a1, a2, a3, . . . an} having n-dimensional information is generated. The matrix is input to a machine learning model using a multiplayer neural network and the matrix is compressed to the two-dimensional information of a matrix {b1, b2}. The machine learning model is a machine learning model obtained by setting, in a multilayer network, a parameter by learning learning data in which n-dimensional information and two-dimensional information are associated with each other. This makes it possible to compress the n-dimensional information to two dimensions and allocate the information to the condition of the matrix {b1, b2}. The approach of expressing the biological information as a matrix of one row and n columns and performing dimensional compression into a matrix of one row and two columns has been described, and any matrix form may be taken according to the calculation model. On the same person, biological information measured for the first time may be represented by a matrix T1{a1, a2, a3, . . . , an}, biological information measured for the second time may be represented by a matrix T2{b1, b2, b3, . . . , bn}, and the matrix T1 and the matrix T2 may be connected. In other words, the first row generates the matrix T1 and the second row generates the matrix Tn. By inputting the matrix Tn to the multiplayer network as in the above-described case, the matrix may be compressed to two-dimensional information and condition allocation may be performed.

### SUMMARY

As described above, the biological information processing apparatus according to the disclosure (for example, the application backend 100) acquires biological information on a subject, generates condition information representing a biological condition of the subject based on the biological information, and registers the condition information in the P2P database 223 (covering a blockchain). More specifically, the biological information processing apparatus generates a condition code representing the biological condition of the subject by encoding the biological information by performing a dimensional compression process and registers condition information containing the condition code in the P2P database 223. Encoding the biological information by the dimensional compression process makes it more difficult to specify the individual and reduce the data size of the condition information. Registering the condition information in the P2P database 223 allows linkage of the condition information with another hospital with authenticity of the condition information being secured. Standardizing the condition information according to given standards allows each hospital to appropriately analyze and utilize the condition information.

The biological information processing apparatus is able to recognize the condition transition pattern of the subject by performing chronological analysis on the condition information using a given method and thus identify the background of the onset of a disease per subject. The biological information processing apparatus generates condition information in consideration of not only diagnostic information from a doctor but also environment information and thus is able to calculate condition information on the subject accurately. Accordingly, the biological information processing apparatus according to the disclosure is able to contribute to realization of individual medicine (or improvement in supportive therapy).

The preferable embodiments of the disclosure have been described in detail with reference to the accompanying drawings; however, the technical scope of the disclosure is not limited to the examples. It is obvious that those with general knowledge in the technical field of the disclosure can reach various modifications or corrections within the scope of the technical idea described in the claims and it is understood that they naturally belong to the technical scope of the disclosure.

The effects described herein are explanatory or exemplary only and thus are not definitive. In other words, the technique according to the disclosure can achieve, together with the above-described effects or instead of the above-described effects, other effects obvious to those skilled in the art from the description herein.

The following configuration also belongs to the technical scope of the disclosure.

(1) A biological information processing method comprising:

acquiring biological information on a subject;

based on the biological information, generating condition information representing a biological condition of the subject; and

registering the condition information in a P2P database.

(2) The biological information processing method according to (1), wherein the condition information contains a condition code that is generated by encoding the biological information using a given method and that represents the biological condition.
(3) The biological information processing method according to (2), wherein the biological information contains at least any one of physical information that is information on a body of the subject and environment information that is information on an environment that affects the subject.
(4) The biological information processing method according to (3), wherein the condition code is generated by, using the given method, encoding at least any one of a physical code that is generated by encoding the physical information using the given method and an environment code that is generated by encoding the environment information using the given method.
(5) The biological information processing method according to (4), wherein the condition information contains, in addition to the condition code, at least any one of the physical code and the environment code.
(6) The biological information processing method according to any one of (2) to (5), wherein the condition information contains, in addition to the condition code, a method code representing the given method.
(7) The biological information processing method according to any one of (2) to (6), wherein the biological information processing method comprises, as the given method, generating the condition code by compressing dimensions of the biological information.
(8) The biological information processing method according to (7), wherein the biological information processing method comprises, compressing the dimensions using at least any one of table conversion and a machine learning approach.
(9) The biological information processing method according to any one of (3) to (5), wherein the physical information contains at least any one of anthropometric information, diagnostic information, treatment information, and operation information on the subject.
(10) The biological information processing method according to any one of (3) to (5), wherein the environment information contains at least any one of information on lifestyle habits, medication information, and information that is acquired using a wearable terminal device that is worn by the subject, which are sets of information on the subject.
(11) The biological information processing method according to any one of (1) to (10), further comprising:

extracting, from time-series data in which the biological information is arranged chronologically, a baseline component representing an irreversible change in the time-series data; and

based on variation in the baseline component, controlling registration of the condition information in the P2P database.

(12) The biological information processing method according to (11), the biological information processing method comprises determining to register the condition information in the P2P database when it is confirmed that the baseline component varies largely with respect to a given threshold from a time of previous registration of the condition information on the subject in the P2P database.
(13) The biological information processing method according to any one of (1) to (12), further comprising:

by comparing the condition information representing the biological condition of the subject with other sets of condition information representing biological conditions of other subjects, extracting a similar subject who had in the past a biological condition similar to that of the subject from the other subjects; and

based on a transition pattern of the biological condition of the similar subject, predicting a transition pattern of a future biological condition of the subject.

(14) The biological information processing method according to (13), further comprising, when it is predicated that the future biological condition of the subject is not preferable, presenting a method of making the biological condition of the subject preferable based on the transition pattern of the biological condition of the similar subject.
(15) The biological information processing method according to any one of (1) to (14), wherein the P2P database is a blockchain.
(16) A biological information processing apparatus comprising:

a biological information acquisition unit configured to acquire biological information on a subject;

a condition information generator configured to generate condition information representing a biological condition of the subject based on the biological information; and

a register configure to register the condition information in a P2P database.

(17) A biological information processing system comprising:

a biological information acquisition unit configured to acquire biological information on a subject;

a condition information generator configured to generate condition information representing a biological condition of the subject based on the biological information; and

a register configure to register the condition information in a P2P database.

(18) A biological information processing method comprising:

acquiring biological information on a subject;

generating condition information representing a biological condition of the subject based on the biological information; and

registering the condition information as data of a distributed network.

### REFERENCE SIGNS LIST

100 APPLICATION BACKEND
110 PROCESSOR
111 BIOLOGICAL INFORMATION ACQUISITION UNIT
112 CONDITION INFORMATION GENERATOR
113 ACCOMPANYING INFORMATION GENERATOR
114 REGISTRATION DETERMINATION UNIT
115 PROPOSAL UNIT
120 STORAGE
130 COMMUNICATION UNIT
140 INPUT UNIT
150 OUTPUT UNIT
200 INTERNAL SERVER
210 PROCESSOR
211 ACQUISITION UNIT
212 TRANSACTION GENERATOR
213 CONSENSUS FORMATION UNIT
220 STORAGE
221 SHARED DATA
222 TRANSACTION STORAGE
223 P2P DATABASE
230 COMMUNICATION UNIT
300 EXTERNAL SERVER
400 INTERNAL NETWORK
500 P2P NETWORK

1. A biological information processing method comprising:
acquiring biological information on a subject;
based on the biological information, generating condition information representing a biological condition of the subject; and
registering the condition information in a P2P database.

2. The biological information processing method according to claim 1, wherein the condition information contains a condition code that is generated by encoding the biological information using a given method and that represents the biological condition.

3. The biological information processing method according to claim 2, wherein the biological information contains at least any one of physical information that is information on a body of the subject and environment information that is information on an environment that affects the subject.

4. The biological information processing method according to claim 3, wherein the condition code is generated by, using the given method, encoding at least any one of a physical code that is generated by encoding the physical information using the given method and an environment code that is generated by encoding the environment information using the given method.

5. The biological information processing method according to claim 4, wherein the condition information contains, in addition to the condition code, at least any one of the physical code and the environment code.

6. The biological information processing method according to claim 2, wherein the condition information contains, in addition to the condition code, a method code representing the given method.

7. The biological information processing method according to claim 2, wherein the biological information processing method comprises, as the given method, generating the condition code by compressing dimensions of the biological information.

8. The biological information processing method according to claim 7, wherein the biological information processing method comprises, compressing the dimensions using at least any one of table conversion and a machine learning approach.

9. The biological information processing method according to claim 3, wherein the physical information contains at least any one of anthropometric information, diagnostic information, treatment information, and operation information on the subject.

10. The biological information processing method according to claim 3, wherein the environment information contains at least any one of information on lifestyle habits, medication information, and information that is acquired using a wearable terminal device that is worn by the subject, which are sets of information on the subject.

11. The biological information processing method according to claim 1, further comprising:
extracting, from time-series data in which the biological information is arranged chronologically, a baseline component representing an irreversible change in the time-series data; and
based on variation in the baseline component, controlling registration of the condition information in the P2P database.

12. The biological information processing method according to claim 11, the biological information processing method comprises determining to register the condition information in the P2P database when it is confirmed that the baseline component varies largely with respect to a given threshold from a time of previous registration of the condition information on the subject in the P2P database.

13. The biological information processing method according to claim 1, further comprising:
by comparing the condition information representing the biological condition of the subject with other sets of condition information representing biological conditions of other subjects, extracting a similar subject who had in the past a biological condition similar to that of the subject from the other subjects; and
based on a transition pattern of the biological condition of the similar subject, predicting a transition pattern of a future biological condition of the subject.

14. The biological information processing method according to claim 13, further comprising, when it is predicated that the future biological condition of the subject is not preferable, presenting a method of making the biological condition of the subject preferable based on the transition pattern of the biological condition of the similar subject.

15. The biological information processing method according to claim 1, wherein the P2P database is a blockchain.

16. A biological information processing apparatus comprising:
a biological information acquisition unit configured to acquire biological information on a subject;
a condition information generator configured to generate condition information representing a biological condition of the subject based on the biological information; and
a register configure to register the condition information in a P2P database.

17. A biological information processing system comprising:
a biological information acquisition unit configured to acquire biological information on a subject;
a condition information generator configured to generate condition information representing a biological condition of the subject based on the biological information; and
a register configure to register the condition information in a P2P database.

18. A biological information processing method comprising:
acquiring biological information on a subject;
generating condition information representing a biological condition of the subject based on the biological information; and
registering the condition information as data of a distributed network.
